(19) United States
(12) Patent Application Publication
(10) Pub. No.: US 2008/0015193 A1
(43) Pub. Date: Jan. 17, 2008

(54) Certain azoles exhibiting ATP-utilizing enzyme inhibitory activity, compositions, and uses thereof

(76) Inventors: Jose Serafin Mendoza, Chapel Hill, NC (US); John K. Dickson JR., Apex, NC (US); Carl Nicholas Hodge, Los Gatos, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/818,914

(22) Filed: Jun. 15, 2007

Related U.S. Application Data
(60) Provisional application No. 60/815,363, filed on Jun. 20, 2006.

(51) Int. Cl. A61K 31/5377; A61K 31/4184; A61K 31/4192; A61K 31/42; A61K 31/428; A61P 1/00; A61P 19/02; A61P 3/10; A61P 35/00; C07D 277/62; C07D 263/02; C07D 249/18; C07D 241/36; C07D 237/02; C07D 235/04; C07D 215/00; C07D 213/00; A61P 43/00; C07D 295/00; A61P 31/00; A61P 25/00; A61P 11/00; A61K 31/4427; A61K 31/47; A61K 31/498; A61K 31/501

(52) U.S. Cl. 514/236.8; 514/249; 514/252.05; 514/256; 514/314; 514/338; 514/340; 514/359; 514/367; 514/374; 514/394; 544/124; 544/235; 544/238; 544/242; 546/152; 546/271.4; 546/273.4; 548/152; 548/235; 548/257; 548/304.7

(57) ABSTRACT

Certain oxazole-based compounds exhibiting ATP-utilizing enzyme inhibitory activity, methods of using compounds exhibiting ATP-utilizing enzyme inhibitory activity, and compositions comprising compounds exhibiting ATP-utilizing enzyme inhibitory activity, are disclosed.

This application claims the benefit of U.S. Provisional Patent Application No. 60/815,363, filed Jun. 20, 2006, which is incorporated herein by reference for all purposes.

ATP-utilizing enzymes catalyze the transfer of a phosphate group from an adenosine triphosphate (ATP) molecule to a biomolecule such as a protein or carbohydrate. Examples of ATP-utilizing enzymes include, but are not limited to, synthetases, ligases, and kinases.

Protein kinases encompass a large family of functionally and structurally related enzymes that are responsible for the control of a wide variety of cellular processes including signal transduction, metabolism, transcription, cell cycle progression, cytoskeletal rearrangement and cell movement, apoptosis, and differentiation. In general, protein kinases control protein activity by catalyzing the addition of a negatively charged phosphate group from a phosphate-containing molecule such as cyclic adenosine monophosphate (cAMP), adenosine diphosphate (ADP), and ATP, to other proteins. Protein phosphorylation in turn can modulate or regulate the functioning of a target protein. Protein phosphorylation is known to play a role in intercellular communication during development, in physiological responses and in homeostasis, and in the functioning of the nervous and immune systems.

Receptor tyrosine kinases are important in the transmission of biochemical signals that initiate cell replication. They are large enzymes that span the cell membrane and possess an extracellular binding domain for growth factors such as epidermal growth factor (EGF), and an intracellular portion that functions as a kinase to phosphorylate tyrosine amino acids in proteins and hence influence cell proliferation. Various classes of receptor tyrosine kinases are known based on families of growth factors that bind to different receptor tyrosine kinases. The classification includes Class I receptor tyrosine kinases comprising the EGF family of receptor tyrosine kinases such as the EGF, TGF.alpha., NEU, erbB, Xmrk, HER and let23 receptors, Class II receptor tyrosine kinases comprising the insulin family of receptor tyrosine kinases such as the insulin, IGFI and insulin-related receptor (IRR) receptors and Class III receptor tyrosine kinases comprising the platelet-derived growth factor (PDGF) family of receptor tyrosine kinases such as the PDGF.alpha., PDGF.beta. and colony-stimulating factor 1 (CSF1) receptors.

A family of type III receptor tyrosine kinases including Flt-3, c-Kit, PDGF-receptor and c-Fms play an important role in the maintenance, growth and development of hematopoietic and non-hematopoietic cells. Flt-3 and c-Kit regulate maintenance of stem cell/early progenitor pools as well the development of mature lymphoid and myeloid cells. Both receptors contain an intrinsic kinase domain that is activated upon ligand-mediated dimerization of the receptors. Upon activation, the kinase domain induces autophosphorylation of the receptor as well as the phosphorylation of various cytoplasmic proteins that help propagate the activation signal leading to growth, differentiation and survival. Some of the downstream regulators of Flt-3 and c-Kit receptor signaling include, PLC.gamma., PI3-kinase, Grb-2, SHIP and Src related kinases. Both receptor tyrosine kinases have been shown to play a role in a variety of hematopoietic and non-hematopoietic malignancies. Mutations that induce ligand independent activation of Flt-3 and c-Kit have been implicated acute-myelogenous leukemia (AML), acute lymphocytic leukemia (ALL), mastocytosis and gastrointestinal stromal tumor (GIST). These mutations include single amino acid changes in the kinase domain or internal tandem duplications, point mutations or in-frame deletions of the juxtamembrane region of the receptors. In addition to activating mutations, ligand dependent (autocrine or paracrine) stimulation of over-expressed wild-type Flt-3 or c-Kit can contribute to the malignant phenotype.

PDGF-receptor (PDGFR) has two subunits-PDGFR-.alpha. and PDGRR-.beta., that can form homo or heterodimers upon ligand binding. There are several PDGF ligands: AB, BB, CC and DD. PDGFR is expressed on early stem cells, mast cells, myeloid cells, mesenchymal cells and smooth muscle cells. Only PDGFR-.beta. has been implicated in myeloid leukemias-usually as a translocation partner with Tel, Huntingtin interacting protein (HIPI) or Rabaptin5. Recently it was shown that activation mutations in PDGFR-.alpha. kinase domain are in gastrointestinal stromal tumors (GIST).

ATP-utilizing enzymes, such as protein kinases, therefore, represent a broad class of pharmacological targets of interest for the treatment of human disease. The identification and development of compounds that selectively inhibit the functioning of ATP-utilizing enzymes is therefore of considerable interest.

Provided is at least one chemical entity chosen from compounds of Formula I: 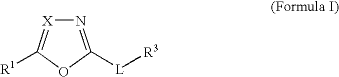
and pharmaceutically acceptable salts, solvates, chelates, non-covalent complexes, prodrugs, and mixtures thereof, wherein
R¹ is chosen from optionally substituted phenyl, optionally substituted furanyl, optionally substituted thienyl, optionally substituted pyridinyl, and optionally substituted quinolinyl;
X is CR²;
R² is hydrogen;
L is chosen from a covalent bond, —CH₂—, —CH═CH—, —CH₂O—, —CH₂NHC(O)—, and —C(O)—, and
R³ is chosen from optionally substituted aryl and optionally substituted heteroaryl, and
   provided that
if R¹ is chosen from optionally substituted phenyl and pyridinyl, and L is chosen from a covalent bond and —CH₂—, then R³ is not chosen from
   optionally substituted benzo[d][1,3]dioxolyl,
   optionally substituted 1,3-dioxoisoindolin-2-yl,
   optionally substituted 1-oxophthalazin-2(1H)-yl,
   optionally substituted 7-oxo-4,5,6,7-tetrahydroindazol-1-yl,
   optionally substituted 5-oxo-5,6,7,8-tetrahydroquinolin-2-yl,
   optionally substituted 1,3-dioxo-1,3-dihydroisobenzofuran-5-yl,
   2,3′-biquinolin-4-yl;
   2,2′-biquinolin-4-yl;
   (isoquinolin-3-yl)quinolin-4-yl;
   quinolin-4-yl;
   2-methyl-3-hydroxy-quinolin-4-yl;
   2-phenyl-quinolin-4-yl;
   quinolin-2-yl;
   quinolin-5-yl;
   optionally substituted thieno[3,2-b]pyridin-2-yl),
   optionally substituted thieno[2,3-b]pyridin-2-yl,
   optionally substituted benzo[d][1,3]dioxole-5-yl,
   optionally substituted 2-oxo-2H-chromen-3-yl, and
   optionally substituted 2-oxo-1,2-dihydroquinolin-4-yl;
if R¹ is optionally substituted phenyl and L is chosen from —CH₂—, —CH═CH—, and —C(O)—, then R³ is not chosen from benzofuran-3-yl and benzo[d]oxazol-2-yl;
if R¹ is optionally substituted phenyl and L is —CH₂O—, then R³ is not quinolin-2-yl;
if R¹ is chosen from optionally substituted phenyl, pyridinyl, thiophenyl, and L is a covalent bond, then R³ is not 6,7-dichloro-3-(4-(pyrrolidin-1-yl)butylamino)quinoxalin-2-yl;
if R¹ is optionally substituted phenyl, and L is a covalent bond, then R³ is not 1H-benzimidazol-5-yl optionally substituted at the 2-position of the benzimidazole ring with a group chosen from optionally substituted cycloalkyl, optionally substituted aryl, optionally substituted heteroaryl, optionally substituted aralkyl, optionally substituted heteroaralkyl, optionally substituted heterocycloalkyl, hydroxyl, alkylthio, and alkylsulfonyl;
   and the compound of Formula I is not chosen from
7-phenyl-3-(5-phenyloxazol-2-yl)-3H-oxazolo[3,2-a][1,3,5]triazine-2,4-dione;
5-(5-phenyloxazol-2-yl)isobenzofuran-1,3-dione;
2-(chroman-6-yl)-5-(pyridin-4-yl)oxazole;
2-(7-(3,4-dichlorophenyl)-5-methyl-4,7-dihydropyrazolo[1,5-a]pyrimidin-6-yl)-5-phenyloxazole;
2-(2-ethylthieno[3,2-d]pyrimidin-4-yl)-5-(4-methoxyphenyl)oxazole;
2-(2-ethylthieno[3,2-d]pyrimidin-4-yl)-5-phenyloxazole;
2-(1,3-dimethyl-1,2,3,4-tetrahydroquinazolin-6-yl)-5-phenyloxazole; and
ethyl 2-morpholino-4-phenyl-6-(5-phenyloxazol-2-yl)-7-propylpyrrolo[1,2-b]pyridazine-5-carboxylate.

Provided is at least one chemical entity chosen from
2-(benzo[d][1,3]dioxol-6-yl)-5-(2-fluorophenyl)-1,3,4-oxadiazole;
2-(4-(benzyloxy)-3-methoxyphenyl)-5-styryl-1,3,4-oxadiazole;
2-(benzo[d][1,3]dioxol-6-yl)-5-(furan-2-yl)-1,3,4-oxadiazole;
2-(4-ethoxyphenyl)-5-(4-fluorophenyl)-1,3,4-oxadiazole;
4-(4-(5-(4-fluorophenyl)-1,3,4-oxadiazol-2-yl)phenyl)morpholine;
4-(5-(4-chlorophenyl)-1,3,4-oxadiazol-2-yl)-N,N-dimethylbenzenamine;
4-(4-(5-(4-methoxyphenyl)-1,3,4-oxadiazol-2-yl)phenyl)morpholine;
3-(5-(3-aminophenyl)-1,3,4-oxadiazol-2-yl)benzenamine;
4-(5-(4-aminophenyl)-1,3,4-oxadiazol-2-yl)benzenamine;
4-(5-(3-methoxyphenyl)-1,3,4-oxadiazol-2-yl)benzenamine;
4-(5-(4-(difluoromethylsulfonyl)phenyl)-1,3,4-oxadiazol-2-yl)-N,N-dimethylbenzenamine;
4-(5-(4-ethoxyphenyl)-1,3,4-oxadiazol-2-yl)pyridine;
N,N-dimethyl-4-(5-(pyridin-3-yl)-1,3,4-oxadiazol-2-yl)benzenamine;
4-(5-(4-butoxyphenyl)-1,3,4-oxadiazol-2-yl)pyridine;
4-(5-(4-isobutoxyphenyl)-1,3,4-oxadiazol-2-yl)pyridine;
4-(5-(3,4-dimethoxyphenyl)-1,3,4-oxadiazol-2-yl)pyridine;
4-(5-(2,4-dichlorophenyl)-1,3,4-oxadiazol-2-yl)pyridine;
4-(5-(pyridin-4-yl)-1,3,4-oxadiazol-2-yl)benzenamine;
4-(5-p-tolyl-1,3,4-oxadiazol-2-yl)pyridine;
4-(5-(2-chlorophenyl)-1,3,4-oxadiazol-2-yl)pyridine;
4-(5-(pyridin-4-yl)-1,3,4-oxadiazol-2-yl)pyridine;
4-(5-phenyl-1,3,4-oxadiazol-2-yl)benzoic acid;
2-(5-phenyl-1,3,4-oxadiazol-2-yl)benzenamine;
2,5-diphenyl-1,3,4-oxadiazole;
3-(5-(2-bromophenyl)-1,3,4-oxadiazol-2-yl)-7-(diethylamino)-2H-chromen-2-one;
3-(5-(2-bromophenyl)-1,3,4-oxadiazol-2-yl)-6-methoxy-2H-chromen-2-one;
3-(5-(furan-2-yl)-1,3,4-oxadiazol-2-yl)-1-methyl-1H-indole;
1-methyl-3-(5-(thiophen-2-yl)-1,3,4-oxadiazol-2-yl)-1H-indole;
2-(3,4,5-trimethoxyphenyl)-5-(5-methylfuran-2-yl)-1,3,4-oxadiazole;
2-(4-methoxyphenyl)-5-(5-methylfuran-2-yl)-1,3,4-oxadiazole;
ethyl 2-(4-(5-(thiophen-2-yl)-1,3,4-oxadiazol-2-yl)phenoxy)acetate; and
3-(4-(5-(furan-2-yl)-1,3,4-oxadiazol-2-yl)phenylcarbamoyl)propanoic acid;
   and pharmaceutically acceptable salts, solvates, chelates, non-covalent complexes, prodrugs, and mixtures thereof.

Provided is a pharmaceutical composition comprising at least one pharmaceutically acceptable vehicle, and a therapeutically effective amount of at least one chemical entity described herein.

Provided is a method of treating at least one disease in a patient in need of such treatment comprising administering to the patient a therapeutically effective amount of at least one chemical entity described herein.

Provided is a method of inhibiting at least one ATP-utilizing enzyme in a subject comprising administering to the subject at least one chemical entity described herein.

Provided is a packaged pharmaceutical formulation comprising a pharmaceutical composition described herein and instructions for using the composition to treat a mammal.

Additional embodiments of the invention are set forth in the description which follows, or may be learned by practice of the invention.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements. At the very least, and not as an attempt to limit the application of the doctrine of equivalents to the scope of the claims, each numerical parameter as set forth in the claims should at least be construed in light of the number of reported significant digits and by applying ordinary rounding techniques.

“Acyl” refers to a radical —C(O)R, where R is hydrogen, alkyl, substituted alkyl, substituted cycloalkyl, substituted heterocycloalkyl, substituted aryl, or substituted heteroaryl group as defined herein. Representative examples include, but are not limited to, formyl, acetyl, cyclohexylcarbonyl, cyclohexylmethylcarbonyl, benzoyl, benzylcarbonyl, and the like.

“Alkanyl” refers to a saturated branched, straight-chain or cyclic alkyl group derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl; butanyls such as butan-1-yl, butan-2-yl (sec-butyl), 2-methyl-propan-1-yl (isobutyl), 2-methyl-propan-2-yl (t-butyl), cyclobutan-1-yl; and the like.

“Alkenyl” refers to an unsaturated branched, straight-chain or cyclic alkyl group having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl; and the like. In certain embodiments, an alkenyl group has from 2 to 20 carbon atoms and in other embodiments, from 2 to 6 carbon atoms.

“Alkoxy” refers to a radical —OR where R represents an alkyl, substituted alkyl, substituted cycloalkyl, substituted heterocycloalkyl, substituted aryl, or substituted heteroaryl group as defined herein. Representative examples include, but are not limited to, methoxy, ethoxy, propoxy, butoxy, cyclohexyloxy, and the like.

“Alkoxycarbonyl” refers to a radical —C(O)— alkoxy where alkoxy is as defined herein.

“Alkyl” refers to a saturated or unsaturated, branched, straight-chain or cyclic monovalent hydrocarbon group derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In certain embodiments, an alkyl group comprises from 1 to 20 carbon atoms. In other embodiments, an alkyl group comprises from 1 to 6 carbon atoms, and is referred to as a lower alkyl group.

“Alkynyl” refers to an unsaturated branched, straight-chain or cyclic alkyl group having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group has from 2 to 20 carbon atoms and in other embodiments, from 3 to 6 carbon atoms.

“Amino” refers to the radical —NH₂.

“Aminocarbonyl” refers to the group —C(O)NRR′ where R and R′ are independently chosen from hydrogen, alkyl, substituted alkyl, substituted cycloalkyl, substituted heterocycloalkyl, substituted aryl, or substituted heteroaryl group as defined herein, or optionally R′ and R″ together with the nitrogen atom to which R and R′ are attached form one or more heterocyclic or substituted heterocyclic rings.

“Aryl” encompasses:
5- and 6-membered carbocyclic aromatic rings, for example, benzene;
bicyclic ring systems wherein at least one ring is carbocyclic and aromatic, for example, naphthalene, indane, and tetralin; and
tricyclic ring systems wherein at least one ring is carbocyclic and aromatic, for example, fluorene.
   For example, aryl includes 5- and 6-membered carbocyclic aromatic rings fused to a 5- to 7-membered heterocycloalkyl ring containing 1 or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings is fused with a heterocycloalkyl aromatic ring, the resulting ring system is heteroaryl, not aryl, as defined herein.

“Arylalkyl” or “aralkyl” refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl, and/or arylalkynyl is used. In certain embodiments, an arylalkyl group can be (C₆₋₃₀) arylalkyl, e.g., the alkanyl, alkenyl or alkynyl moiety of the arylalkyl group can be (C₁₋₁₀) and the aryl moiety can be (C₆₋₂₀).

“Aryloxycarbonyl” refers to a radical —C(O)—O—R wherein R is chosen from aryl and substituted aryl as defined herein.

“Carbonyl” refers to the radical —C(O).

“Carboxy” refers to the radical —C(O)OH.

“Cleave” refers to breakage of chemical bonds and is not limited to chemical or enzymatic reactions or mechanisms unless clearly indicated by the context.

The term “chelate” refers to the chemical entity formed by the coordination of a compound to a metal ion at two (or more) points.

The term “non-covalent complex” refers to the chemical entity formed by the interaction of a compound and another molecule wherein a covalent bond is not formed between the compound and the molecule. For example, complexation can occur through van der Waals interactions, hydrogen bonding, and electrostatic interactions (also called ionic bonding).

As noted above, prodrugs also fall within the scope of chemical entities, for example ester or amide derivatives of the compounds of Formula I. The term “prodrugs” includes any compounds that become compounds of Formula I when administered to a patient, e.g., upon metabolic processing of the prodrug. Examples of prodrugs include, but are not limited to, acetate, formate, and benzoate and like derivatives of functional groups (such as alcohol or amine groups) in the compounds of Formula I.

The term “solvate” refers to the compound formed by the interaction of a solvent and a compound. Suitable solvates are pharmaceutically acceptable solvates, such as hydrates, including monohydrates and hemi-hydrates.

“Bond” refers to a covalent attachment between two atoms.

“Cyano” refers to the radical —CN.

“Cycloalkyl” refers to a saturated or unsaturated (although not aromatic) mono- or bicyclic alkyl group. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In certain embodiments, the cycloalkyl group can be C₃₋₁₀ cycloalkyl, such as, for example, C₃₋₆ cycloalkyl.

“Disease” refers to any disease, disorder, condition, symptom, or indication.

“Enzyme” refers to any naturally occurring or synthetic macromolecular substance composed wholly or largely of protein, that catalyzes, more or less specifically, one or more biochemical reactions. The substances upon which the enzyme acts are referred to “substrates,” for which the enzyme possesses a specific binding or “active site,” or “catalytic domain.” Enzymes can also act on macromolecular structures such as muscle fibers.

“Extended release” refers to dosage forms that provide for the delayed, slowed, over a period of time, continuous, discontinuous, or sustained release of the chemical entities of the present disclosure.

“Halogen” or “halo” refers to a fluoro, chloro, bromo, or iodo group.

“Heteroaryl” encompasses:
5- to 7-membered aromatic, monocyclic rings containing one or more, for example, from 1 to 4, or in certain embodiments, from 1 to 3, heteroatoms chosen from N, O, and S, with the remaining ring atoms being carbon; and
bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring.
   For example, heteroaryl includes a 5- to 7-membered heterocycloalkyl aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Examples of heteroaryl groups include, but are not limited to, (as numbered from the linkage position assigned priority 1), 2-pyridyl, 3-pyridyl, 4-pyridyl, 2,3-pyrazinyl, 3,4-pyrazinyl, 2,4-pyrimidinyl, 3,5-pyrimidinyl, 2,3-pyrazolinyl, 2,4-imidazolinyl, isoxazolinyl, oxazolinyl, thiazolinyl, thiadiazolinyl, tetrazolyl, thienyl, benzothiophenyl, furanyl, benzofuranyl, benzoimidazolinyl, indolinyl, pyridizinyl, triazolyl, quinolinyl, pyrazolyl, and 5,6,7,8-tetrahydroisoquinoline. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl as defined above. In certain embodiments, heteroaryl groups can be those derived from thiophene, pyrrole, benzothiophene, benzofuran, indole, pyridine, quinoline, imidazole, oxazole, pyrazine, benzothiazole, isoxazole, thiadiazole, and thiazole.

“Heteroarylalkyl” or “heteroaralkyl” refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl, and/or heteroarylalkynyl is used. In certain embodiments, the heteroarylalkyl group can be a 6 to 30 membered heteroarylalkyl, e.g., the alkanyl, alkenyl or alkynyl moiety of the heteroarylalkyl can be 1 to 10 membered and the heteroaryl moiety can be a 5 to 20-membered heteroaryl.

By “heterocycloalkyl” is meant a single aliphatic ring, usually with 3 to 7 ring atoms, containing at least 2 carbon atoms in addition to 1-3 heteroatoms independently selected from oxygen, sulfur, and nitrogen, as well as combinations comprising at least one of the foregoing heteroatoms. Suitable heterocycloalkyl groups include, for example (as numbered from the linkage position assigned priority 1), 2-pyrrolinyl, 2,4-imidazolidinyl, 2,3-pyrazolidinyl, 2-piperidyl, 3-piperidyl, 4-piperidyl, and 2,5-piperizinyl. Morpholinyl groups are also contemplated, including 2-morpholinyl and 3-morpholinyl (numbered wherein the oxygen is assigned priority 1). Substituted heterocycloalkyl also includes ring systems substituted with one or more oxo (=0) or oxide (—O⁻) substituents, such as piperidinyl N-oxide, morpholinyl-N-oxide, 1-oxo-1-thiomorpholinyl and 1,1-dioxo-1-thiomorpholinyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not-aromatic.

“Leaving group” refers to an atom or a group capable of being displaced by a nucleophile and includes halogen, such as chloro, bromo, fluoro, and iodo, alkoxycarbonyl (e.g., acetoxy), aryloxycarbonyl, mesyloxy, tosyloxy, trifluoromethanesulfonyloxy, aryloxy (e.g., 2,4-dinitrophenoxy), methoxy, N,O-dimethylhydroxylamino, and the like.

“Optional” or “optionally” means that the subsequently described event or circumstance may but need not occur, and that the description includes instances where the event or circumstance occurs and instances in which the event does not.

“Pharmaceutically acceptable” refers to approved or approvable by a regulatory agency of the Federal or a state government or listed in the U.S. Pharmacopeia or other generally recognized pharmacopeia for use in animals, and more particularly in humans.

“Pharmaceutically acceptable salt” refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like.

“Pharmaceutically acceptable excipient, carrier or adjuvant” refers to an excipient, carrier or adjuvant that can be administered to a subject, together with at least one chemical entity of the present disclosure, and which does not destroy the pharmacological activity thereof and is nontoxic when administered in doses sufficient to deliver a therapeutic amount of the compound.

“Pharmaceutically acceptable vehicle” refers to a diluent, adjuvant, excipient or carrier with which at least one chemical entity of the present disclosure is administered.

“Prodrug” refers to a derivative of a therapeutically effective compound that requires a transformation within the body to produce the therapeutically effective compound. Prodrugs can be pharmacologically inactive until converted to the parent compound.

“Promoiety” refers to a form of protecting group that when used to mask a functional group within a drug molecule converts the drug into a prodrug. For example, the promoiety can be attached to the drug via bond(s) that are cleaved by enzymatic or non-enzymatic means in vivo.

“Protecting group” refers to a grouping of atoms that when attached to a reactive group in a molecule masks, reduces or prevents that reactivity. Examples of protecting groups can be found in Green et al., “Protective Groups in Organic Chemistry,” (Wiley, 2nd ed. 1991) and Harrison et al., “Compendium of Synthetic Organic Methods,” Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”), and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Protein kinase,” “kinase,” and “human protein kinase” refer to any enzyme that phosphorylates one or more hydroxyl or phenolic groups in proteins, ATP being the phosphoryl-group donor.

“Stereoisomer” refers to an isomer that differs in the arrangement of the constituent atoms in space. Stereoisomers that are mirror images of each other and optically active are termed “enantiomers,” and stereoisomers that are not mirror images of one another are termed “diastereoisomers.”

“Subject” includes mammals, such as humans. The terms “human” and “subject” are used interchangeably herein.

“Substituted” refers to a group in which one or more hydrogen atoms are each independently replaced with the same or different substituent(s). Typical substituents include, but are not limited to, —X, —R³³, —O⁻, ═O, —OR³³, —SR³³, —S⁻, ═S, —NR³³R³⁴, ═NR³³, —CX₃, —CF₃, —CN, —OCN, —SCN, —NO, —NO₂, ═N₂, —N₃, —S(O)₂O⁻, —S(O)₂OH, —S(O)₂R³³, —OS(O₂)O⁻, —OS(O)₂R³³, —P(O)(O⁻)₂, —P(O)(OR³³)(O⁻), —OP(O)(OR³³)(OR³⁴), —C(O)R³³, —C(S)R³³, —C(O)OR³³, —C(O)NR³³R³⁴, —C(O)O⁻, —C(S)OR³³, —NR³⁵C(O)NR³³R³⁴, —NR³⁵C(S)NR³³R³⁴, —NR³⁵C(NR³³)NR³³R³⁴, —C(NR³³)NR³³R³⁴, —S(O)₂NR³³R³⁴, —NR³⁵S(O)₂R³³, —NR³⁵C(O)R³³, and —S(O)R³³ where each X is independently a halogen; each R³³ and R³⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, —NR³⁵R³⁶, —(O)R³⁵ or —S(O)₂R³⁵ or optionally R³³ and R³⁴ together with the atom to which R³³ and R³⁴ are attached form one or more cycloheteroalkyl, substituted cycloheteroalkyl, heteroaryl, or substituted heteroaryl rings; and R³⁵ and R³⁶ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl or substituted heteroarylalkyl, or optionally R³⁵ and R³⁶ together with the nitrogen atom to which R³⁵ and R³⁶ are attached form one or more cycloheteroalkyl, substituted cycloheteroalkyl, heteroaryl, or substituted heteroaryl rings. In certain embodiments, a tertiary amine or aromatic nitrogen may be substituted with one or more oxygen atoms to form the corresponding nitrogen oxide.

In certain embodiments, substituted aryl and substituted heteroaryl include one or more of the following substituent groups: F, Cl, Br, C₁₋₃ alkyl, substituted alkyl, C₁₋₃ alkoxy, —S(O)₂NR³³R³⁴, —NR³³R³⁴, —CF₃, —OCF₃, —CN, —NR³⁵S(O)₂R³³, —NR³⁵C(O)R³³, C₅₋₁₀ aryl, substituted C₅₋₁₀ aryl, C₅₋₁₀ heteroaryl, substituted C₅₋₁₀ heteroaryl, —C(O)OR³³, —NO₂, —C(O)R³³, —C(O)NR³³R³⁴, —OCHF₂, C₁₋₃ acyl, —SR³³, —S(O)₂OH, —S(O)₂R³³, —S(O)R³³, —C(S)R³³, —C(O)O⁻, —C(S)OR³³, —NR³⁵C(O)NR³³R³⁴, —NR³⁵C(S)NR³³R³⁴, and —C(NR³⁵)NR³³R³⁴, C₃₋₈ cycloalkyl, and substituted C₃₋₈ cycloalkyl, C₃₋₈ heterocycloalkyl, and substituted C₃₋₈ heterocycloalkyl, as defined herein.

In certain embodiments, substituted arylalkyl, and substituted heteroarylalkyl include one or more of the following substitute groups: F, Cl, Br, C₁₋₃ alkyl, C₁₋₃ alkoxy, —S(O)₂NR³³R³⁴, —NR³³R³⁴, —CF₃, —OCF₃, CN, —NR³⁵S(O)₂R³³, —NR³⁵C(O)R³³, C₅₋₁₀ aryl, substituted alkyl, substituted C₅₋₁₀ aryl, C₅₋₁₀ heteroaryl, substituted C₅₋₁₀ heteroaryl, —C(O)OR³³, —NO₂, —C(O)R³³, —C(O)NR³³R³⁴, —OCHF₂, C₁₋₃ acyl, —SR³³, —S(O)₂OH, —S(O)₂R³³, —S(O)R³³, —C(S)R³³, —C(O)O⁻, —C(S)OR³³, —NR³⁵C(O)NR³³R³⁴, —NR³⁵C(S)NR³³R³⁴ and —C(NR³⁵)NR³³R³⁴, C₃₋₈ cycloalkyl, and substituted C₃₋₈ cycloalkyl, as defined herein.

In certain embodiments, substituted alkyl includes one or more of the following substitute groups: C₁₋₃ alkoxy, —NR³³R³⁴, substituted C₅₋₁₀ heteroaryl, —SR³³, C₁₋₃ alkoxy, —S(O)₂NR³³R³⁴, CN, F, Cl, —CF₃, —OCF₃, —NR³⁵S(O)₂R³³, —NR³⁵C(O)R³³, C₅₋₁₀ aryl, substituted C₅₋₁₀ aryl, C₅₋₁₀ heteroaryl, substituted C₅₋₁₀ heteroaryl, —C(O)OR³³, —NO₂, —C(O)R³³, —C(O)NR³³R³⁴, —OCHF₂, C₁₋₃ acyl, —S(O)₂OH, —S(O)₂R³³, —S(O)R³³, —C(S)R, —C(O)O⁻, —C(S)OR³³, —NR³⁵C(O)NR³³NR³⁴, —NR³⁵C(S)NR³³R³⁴, and —C(NR³⁵)NR³³R³⁴, C₃₋₈ cycloalkyl, and substituted C₃₋₈ cycloalkyl, as defined herein.

In certain embodiments, substituted alkenyl includes one or more of the following substitute groups: C₁₋₈ alkyl, substituted C₁₋₈ alkyl, C₅₋₁₀ aryl, substituted C₅₋₁₀ aryl, C₅₋₁₀ heteroaryl, substituted C₅₋₁₀ heteroaryl, C₃₋₈ cycloalkyl, substituted C₃₋₈ cycloalkyl, cycloheteroalkylalkyl, and substituted cycloheteroalkylalkyl, as defined herein.

The term “substituted amino” refers to the group —NHR^{d} or —NR^{d}R^{d} where each R^{d} is independently chosen from: alkyl, substituted alkyl, cycloalkyl, substituted cycloalkyl, acyl, substituted acyl, aryl, substituted aryl, heteroaryl, substituted heteroaryl, heterocycloalkyl, substituted heterocycloalkyl, alkoxycarbonyl, and sulfonyl. Representative examples include, but are not limited to, dimethylamino, methylethylamino, di-(1-methylethyl)amino, (cyclohexyl)(methyl)amino, (cyclohexyl)(ethyl)amino, (cyclohexyl)(propyl)amino, and the like.

“Sulfonyl” refers to a radical —S(O)₂R where R is an alkyl, substituted alkyl, substituted cycloalkyl, substituted heterocycloalkyl, substituted aryl, or substituted heteroaryl group as defined herein. Representative examples include, but are not limited to methylsulfonyl, ethylsulfonyl, propylsulfonyl, butylsulfonyl, and the like.

“Sulfinyl” refers to a radical —S(O)R where R is an alkyl, substituted alkyl, substituted cycloalkyl, substituted heterocycloalkyl, substituted aryl, or substituted heteroaryl group as defined herein. Representative examples include, but are not limited to, methylsulfinyl, ethylsulfinyl, propylsulfinyl, butylsulfinyl, and the like.

“Sulfanyl” refers to a radical —SR where R is an alkyl, substituted alkyl, substituted cycloalkyl, substituted heterocycloalkyl, substituted aryl, or substituted heteroaryl group as defined herein. Representative examples include, but are not limited to, methylthio, ethylthio, propylthio, butylthio, and the like.

“Therapeutically effective amount” refers to the amount of a compound that, when administered to a subject for treating a disease, or at least one of the clinical symptoms of a disease or disorder, is sufficient to affect such treatment for the disease, disorder, or symptom. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, and inhibit at least one physical parameter which may not be discernible to the subject. Further, “treating” or “treatment” refers to delaying the onset of the disease or disorder or at least symptoms thereof in a subject which may be exposed to or predisposed to a disease or disorder even though that subject does not yet experience or display symptoms of the disease or disorder.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

In the specification and the appended claims, the singular forms “a,” “an,” and “the” include plural reference unless the context clearly dictates otherwise.

The compounds of Formula I can be named and numbered in the manner (e.g., using ChemDraw 8.0 or 9.0 Struct=Name algorithm) described below. For example, the compound: 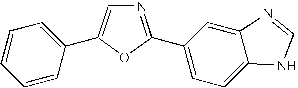
can be named 5-(5-phenyloxazol-2-yl)-1H-benzo[d]imidazole.

Provided is at least one chemical entity chosen from compounds of Formula I: 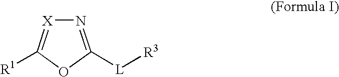
and pharmaceutically acceptable salts, solvates, chelates, non-covalent complexes, prodrugs, and mixtures thereof, wherein
R¹ is chosen from optionally substituted phenyl, optionally substituted furanyl, optionally substituted thienyl, optionally substituted pyridinyl, and optionally substituted quinolinyl;
X is CR²;
R² is hydrogen;
L is chosen from a covalent bond, —CH₂—, —CH═CH—, —CH₂O—, —CH₂NHC(O)—, and —C(O)—, and
R³ is chosen from optionally substituted aryl and optionally substituted heteroaryl, and
   provided that
if R¹ is chosen from optionally substituted phenyl and pyridinyl, and L is chosen from a covalent bond and —CH₂—, then R³ is not chosen from
   optionally substituted benzo[d][1,3]dioxolyl,
   optionally substituted 1,3-dioxoisoindolin-2-yl,
   optionally substituted 1-oxophthalazin-2(1H)-yl,
   optionally substituted 7-oxo-4,5,6,7-tetrahydroindazol-1-yl,
   optionally substituted 5-oxo-5,6,7,8-tetrahydroquinolin-2-yl,
   optionally substituted 1,3-dioxo-1,3-dihydroisobenzofuran-5-yl,
   2,3′-biquinolin-4-yl;
   2,2′-biquinolin-4-yl;
   (isoquinolin-3-yl)quinolin-4-yl;
   quinolin-4-yl;
   2-methyl-3-hydroxy-quinolin-4-yl;
   2-phenyl-quinolin-4-yl;
   quinolin-2-yl;
   quinolin-5-yl;
   optionally substituted thieno[3,2-b]pyridin-2-yl),
   optionally substituted thieno[2,3-b]pyridin-2-yl,
   optionally substituted benzo[d][1,3]dioxole-5-yl,
   optionally substituted 2-oxo-2H-chromen-3-yl, and
   optionally substituted 2-oxo-1,2-dihydroquinolin-4-yl;
if R¹ is optionally substituted phenyl and L is chosen from —CH₂—, —CH═CH—, and —C(O)—, then R³ is not chosen from benzofuran-3-yl and benzo[d]oxazol-2-yl;
if R¹ is optionally substituted phenyl and L is —CH₂O—, then R³ is not quinolin-2-yl;
if R¹ is chosen from optionally substituted phenyl, pyridinyl, thiophenyl, and L is a covalent bond, then R³ is not 6,7-dichloro-3-(4-(pyrrolidin-1-yl)butylamino)quinoxalin-2-yl;
if R¹ is optionally substituted phenyl, and L is a covalent bond, then R³ is not 1H-benzimidazol-5-yl optionally substituted at the 2-position of the benzimidazole ring with a group chosen from optionally substituted cycloalkyl, optionally substituted aryl, optionally substituted heteroaryl, optionally substituted aralkyl, optionally substituted heteroaralkyl, optionally substituted heterocycloalkyl, hydroxyl, alkylthio, and alkylsulfonyl;
and the compound of Formula I is not chosen from
7-phenyl-3-(5-phenyloxazol-2-yl)-3H-oxazolo[3,2-a][1,3,5]triazine-2,4-dione;
5-(5-phenyloxazol-2-yl)isobenzofuran-1,3-dione;
2-(chroman-6-yl)-5-(pyridin-4-yl)oxazole;
2-(7-(3,4-dichlorophenyl)-5-methyl-4,7-dihydropyrazolo[1,5-a]pyrimidin-6-yl)-5-phenyloxazole;
2-(2-ethylthieno[3,2-d]pyrimidin-4-yl)-5-(4-methoxyphenyl)oxazole;
2-(2-ethylthieno[3,2-d]pyrimidin-4-yl)-5-phenyloxazole;
2-(1,3-dimethyl-1,2,3,4-tetrahydroquinazolin-6-yl)-5-phenyloxazole; and
ethyl 2-morpholino-4-phenyl-6-(5-phenyloxazol-2-yl)-7-propylpyrrolo[1,2-b]pyridazine-5-carboxylate.

In certain embodiments, R¹ is chosen from optionally substituted phenyl and optionally substituted pyridinyl.

In certain embodiments, R¹ is chosen from phenyl and pyridinyl, each of which is optionally substituted with one, two or three groups chosen from are selected from halo, cyano, hydroxy, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl.

In certain embodiments, R¹ is chosen from phenyl and pyridinyl, each of which is optionally substituted with one, two or three groups chosen from are selected from optionally substituted lower alkyl, optionally substituted lower alkoxy, halo, hydroxy, and cyano.

In certain embodiments, R¹ is chosen from phenyl and pyridinyl, each of which is optionally substituted with one, two or three groups chosen from lower alkyl, lower alkoxy, halo, hydroxy, and cyano.

In certain embodiments, R¹ is chosen from phenyl and pyridinyl.

In certain embodiments, L is a covalent bond.

In certain embodiments, R³ is chosen from fused 9 or 10 membered heterobicyclic ring systems containing one, two, three, or four heteroatoms chosen from nitrogen, oxygen, and sulfur wherein at least one of the rings in the ring system is aromatic and wherein the ring system is optionally substituted with one, two, or three groups chosen from halo, cyano, hydroxy, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl.

In certain embodiments, R³ is chosen from
phenyl,
pyridinyl,
phenyl substituted with one, two, or three groups chosen from halo, cyano, hydroxy, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl; and
pyridinyl substituted with one, two, or three groups chosen from halo, cyano, hydroxy, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl.

Also provided is at least one chemical entity chosen from compounds of Formula II 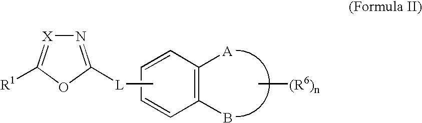
and pharmaceutically acceptable salts, solvates, chelates, non-covalent complexes, prodrugs, and mixtures thereof, wherein R¹, X, and L (which may be on either or both of the rings of the bicyclic ring system) are as described for compounds of Formula I and wherein
-A-B- is chosen from:
(a) —CH═CH—N═CH—,
(b) —CH═CH—CH═N—,
(c) —CH═CH—N═N—,
(d) —CH═N—N═CH—,
(e) —CH═N—CH═N—,
(f) —N═CH—CH═N—,
(g) —CH═CH—NH—,
(h) —CH═CH—O—,
(i) —CH═CH—S—,
(j) —N═CH—NH—,
(k) —CH═N—NH—
(l) —O—CH═N—,
(m) —CH═N—O—,
(n) —S—CH═N—,
(o) —CH═N—S—,
(p) —N═N—NH—,
(q) —CH₂—CH₂—CH═N—,
(r) —CH₂—CH₂—CH₂—NH—,
(s) —CH₂—CH₂—N═CH—,
(t) —CH₂—CH₂—NH—CH₂—,
(u) —CH₂—NH—C(O)—NH—,
(v) —CH₂—O—C(O)—NH—,
(w) —CH₂—NH—S(O)—NH—,
(x) —CH₂—NH—SO₂—NH—,
(y) —CH₂—CH₂—C(O)—NH—, and
(z) —CH═CH—C(O)—NH—;
n is chosen from 0, 1, 2, and 3; and
R⁶ (which may be on either or both of the rings of the heterobicyclic ring system) is chosen from halo, cyano, hydroxy, oxo, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl, provided that
if R¹ is chosen from optionally substituted phenyl and pyridinyl, and L is chosen from a covalent bond and —CH₂—, then R³ is not chosen from
2,3′-biquinolin-4-yl;
2,2′-biquinolin-4-yl;
(isoquinolin-3-yl)quinolin-4-yl;
quinolin-4-yl;
2-methyl-3-hydroxy-quinolin-4-yl;
2-phenyl-quinolin-4-yl;
quinolin-2-yl; and
quinolin-5-yl;
if R¹ is optionally substituted phenyl and L is —CH₂O—, then R³ is not quinolin-2-yl;
if R¹ is chosen from optionally substituted phenyl, pyridinyl, thiophenyl, and L is a covalent bond, then R³ is not 6,7-dichloro-3-(4-(pyrrolidin-1-yl)butylamino)quinoxalin-2-yl;
if R¹ is optionally substituted phenyl, and L is a covalent bond, then R³ is not 1H-benzimidazol-5-yl optionally substituted at the 2-position of the benzimidazole ring with a group chosen from optionally substituted cycloalkyl, optionally substituted aryl, optionally substituted heteroaryl, optionally substituted aralkyl, optionally substituted heteroaralkyl, optionally substituted heterocycloalkyl, hydroxyl, alkylthio, and alkylsulfonyl; and
if R¹ is optionally substituted phenyl and L is chosen from —CH₂—, —CH═CH—, and —C(O)—, then R³ is not chosen from benzofuran-3-yl and benzo[d]oxazol-2-yl.

In certain embodiments, L is bound to the phenyl ring.

In certain embodiments, -A-B- is chosen from

—N═CH—NH—,

—S—CH═N—,

—CH═CH—CH═N—,

—N═N—NH—,

—CH═CH—N═CH—, and

—N═CH—CH═N—.

Also provided is at least one chemical entity chosen from compounds of Formula III 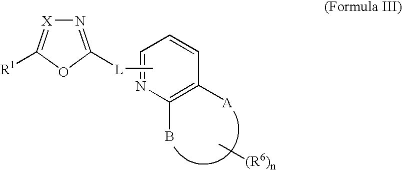
and pharmaceutically acceptable salts, solvates, chelates, non-covalent complexes, prodrugs, and mixtures thereof, wherein R¹, X, and L (which may be on either or both of the rings of the bicyclic ring system) are as described for compounds of Formula I and wherein
-A-B- is chosen from:
(a) —CH═CH—N═CH—,
(b) —CH═CH—CH═N—,
(c) —CH═CH—N═N—,
(d) —CH═N—N═CH—,
(e) —CH═N—CH═N—,
(f) —N═CH—CH═N—,
(g) —CH═CH—NH—,
(h) —CH═CH—O—,
(i) —CH═CH—S—,
(j) —N═CH—NH—,
(k) —CH═N—NH—
(l) —O—CH═N—,
(m) —CH═N—O—,
(n) —S—CH═N—,
(o) —CH═N—S—,
(p) —N═N—NH—,
(q) —CH₂—CH₂—CH═N—,
(r) —CH₂—CH₂—CH₂—NH—,
(s) —CH₂—CH₂—N═CH—,
(t) —CH₂—CH₂—NH—CH₂—,
(u) —CH₂—NH—C(O)—NH—,
(v) —CH₂—O—C(O)—NH—,
(w) —CH₂—NH—S(O)—NH—,
(x) —CH₂—NH—SO₂—NH—,
(y) —CH₂—CH₂—C(O)—NH—,
(z) —CH═CH—C(O)—NH—, and
(aa) —CH═CH—CH═CH—,
n is chosen from 0, 1, 2, and 3; and
R⁶ (which may be on either or both of the rings of the heterobicyclic ring system) is chosen from halo, cyano, hydroxy, oxo, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl, provided that
if R¹ is chosen from optionally substituted phenyl and pyridinyl, and L is chosen from a covalent bond and —CH₂—, then R³ is not chosen from
   2,3′-biquinolin-4-yl;
   2,2′-biquinolin-4-yl;
   (isoquinolin-3-yl)quinolin-4-yl;
   quinolin-4-yl;
   2-methyl-3-hydroxy-quinolin-4-yl;
   2-phenyl-quinolin-4-yl;
   quinolin-2-yl;
   quinolin-5-yl;
   optionally substituted thieno[3,2-b]pyridin-2-yl, and
   optionally substituted thieno[2,3-b]pyridin-2-yl; and
if R¹ is optionally substituted phenyl and L is —CH₂O—, then R³ is not quinolin-2-yl.

In certain embodiments, L is bound to the pyridinyl ring.

In certain embodiments, -A-B- is —CH═CH—CH═CH—.

Also provided is at least one chemical entity chosen from compounds of Formula IV 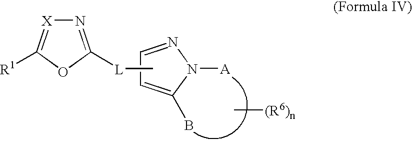
and pharmaceutically acceptable salts, solvates, chelates, non-covalent complexes, prodrugs, and mixtures thereof, wherein R¹, X, and L (which may be on either or both of the rings of the bicyclic ring system) are as described for compounds of Formula I and wherein
-A-B- is chosen from:
(a) —CH═CH—N═CH—,
(b) —CH═CH—CH═N—,
(c) —CH═CH—N═N—,
(d) —CH═N—N═CH—,
(e) —CH═N—CH═N—,
(f) —N═CH—CH═N—,
(g) —CH═CH—NH—,
(h) —CH═CH—O—,
(i) —CH═CH—S—,
(j) —N═CH—NH—,
(k) —CH═N—NH—
(l) —O—CH═N—,
(m) —CH═N—O—,
(n) —S—CH═N—,
(o) —CH═N—S—,
(p) —N═N—NH—,
(q) —CH₂—CH₂—CH═N—,
(r) —CH₂—CH₂—CH₂—NH—,
(s) —CH₂—CH₂—N═CH—,
(t) —CH₂—CH₂—NH—CH₂—,
(u) —CH₂—NH—C(O)—NH—,
(v) —CH₂—O—C(O)—NH—,
(w) —CH₂—NH—S(O)—NH—,
(x) —CH₂—NH—SO₂—NH—,
(y) —CH₂—CH₂—C(O)—NH—,
(z) —CH═CH—C(O)—NH—;
(aa) —CH═CH—CH═CH—,
n is chosen from 0, 1, 2, and 3; and
R⁶ (which may be on either or both of the rings of the heterobicyclic ring system) is chosen from halo, cyano, hydroxy, oxo, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl.

In certain embodiments, L is bound to the pyrazole ring.

In certain embodiments, -A-B- is —CH═CH—CH═CH—.

Also provided is at least one chemical entity chosen from compounds of Formula V 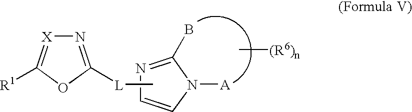
and pharmaceutically acceptable salts, solvates, chelates, non-covalent complexes, prodrugs, and mixtures thereof, wherein R¹, X, and L (which may be on either or both of the rings of the bicyclic ring system) are as described for compounds of Formula I and wherein
-A-B- is chosen from:
(a) —CH═CH—N═CH—,
(b) —CH═CH—CH═N—,
(c) —CH═CH—N═N—,
(d) —CH═N—N═CH—,
(e) —CH═N—CH═N—,
(f) —N═CH—CH═N—,
(g) —CH═CH—NH—,
(h) —CH═CH—O—,
(i) —CH═CH—S—,
(j) —N═CH—NH—,
(k) —CH═N—NH—
(l) —O—CH═N—,
(m) —CH═N—O—,
(n) —S—CH═N—,
(o) —CH═N—S—,
(p) —N═N—NH—,
(q) —CH₂—CH₂—CH═N—,
(r) —CH₂—CH₂—CH₂—NH—,
(s) —CH₂—CH₂—N═CH—,
(t) —CH₂—CH₂—NH—CH₂—,
(u) —CH₂—NH—C(O)—NH—,
(v) —CH₂—O—C(O)—NH—,
(w) —CH₂—NH—S(O)—NH—,
(x) —CH₂—NH—SO₂—NH—,
(y) —CH₂—CH₂—C(O)—NH—,
(z) —CH═CH—C(O)—NH—, and
(aa) —CH═CH—CH═CH—,
n is chosen from 0, 1, 2, and 3; and
R⁶ (which may be on either or both of the rings of the heterobicyclic ring system) is chosen from halo, cyano, hydroxy, oxo, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl.

In certain embodiments, L is bound to the imidazole ring.

In certain embodiments, -A-B- is —CH═CH—CH═N—.

Also provided is at least one chemical entity chosen from compounds of Formula VI 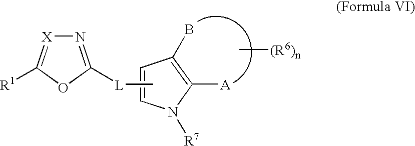
and pharmaceutically acceptable salts, solvates, chelates, non-covalent complexes, prodrugs, and mixtures thereof, wherein R¹, X, and L (which may be on either or both of the rings of the bicyclic ring system) are as described for compounds of Formula I and wherein
-A-B- is chosen from:
(a) —CH═CH—N═CH—,
(b) —CH═CH—CH═N—,
(c) —CH═CH—N═N—,
(d) —CH═N—N═CH—,
(e) —CH═N—CH═N—,
(f) —N═CH—CH═N—,
(g) —CH═CH—NH—,
(h) —CH═CH—O—,
(i) —CH═CH—S—,
(j) —N═CH—NH—,
(k) —CH═N—NH—
(l) —O—CH═N—,
(m) —CH═N—O—,
(n) —S—CH═N—,
(o) —CH═N—S—,
(p) —N═N—NH—,
(q) —CH₂—CH₂—CH═N—,
(r) —CH₂—CH₂—CH₂—NH—,
(s) —CH₂—CH₂—N═CH—,
(t) —CH₂—CH₂—NH—CH₂—,
(u) —CH₂—NH—C(O)—NH—,
(v) —CH₂—O—C(O)—NH—,
(w) —CH₂—NH—S(O)—NH—,
(x) —CH₂—NH—SO₂—NH—,
(y) —CH₂—CH₂—C(O)—NH—,
(z) —CH═CH—C(O)—NH—; and
(aa) —CH═CH—CH═CH—,
n is chosen from 0, 1, 2, and 3; and
R⁶ (which may be on either or both of the rings of the heterobicyclic ring system) is chosen from halo, cyano, hydroxy, oxo, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl and,
R⁷ is chosen from hydrogen, optionally substituted alkyl, optionally substituted cycloalkyl, optionally substituted heterocycloalkyl, optionally substituted aryl, and optionally substituted heteroaryl.

In certain embodiments, L is bound to the pyrrole ring.

In certain embodiments, -A-B- is —CH═CH—CH═CH—.

Also provided is at least one chemical entity chosen from compounds of Formula VII 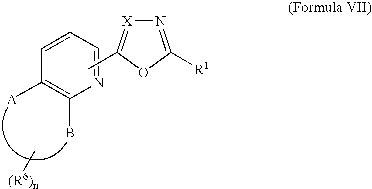
and pharmaceutically acceptable salts, solvates, chelates, non-covalent complexes, prodrugs, and mixtures thereof, wherein R¹ and X are as described for compounds of Formula I and wherein
-A-B- is chosen from:
(a) —CH═CH—N═CH—,
(b) —CH═CH—CH═N—,
(c) —CH═CH—N═N—,
(d) —CH═N—N═CH—,
(e) —CH═N—CH═N—,
(f) —N═CH—CH═N—,
(g) —CH═CH—NH—,
(h) —CH═CH—O—,
(i) —CH═CH—S—,
(j) —N═CH—NH—,
(k) —CH═N—NH—
(l) —O—CH═N—,
(m) —CH═N—O—,
(n) —S—CH═N—,
(o) —CH═N—S—,
(p) —N═N—NH—,
(q) —CH₂—CH₂—CH═N—,
(r) —CH₂—CH₂—CH₂—NH—,
(s) —CH₂—CH₂—N═CH—,
(t) —CH₂—CH₂—NH—CH₂—,
(u) —CH₂—NH—C(O)—NH—,
(v) —CH₂—O—C(O)—NH—,
(w) —CH₂—NH—S(O)—NH—,
(x) —CH₂—NH—SO₂—NH—,
(y) —CH₂—CH₂—C(O)—NH—,
(z) —CH═CH—C(O)—NH—, and
(aa) —CH═CH—CH═CH—,
n is chosen from 0, 1, 2, and 3; and
R⁶ (which may be on either or both of the rings of the heterobicyclic ring system) is chosen from halo, cyano, hydroxy, oxo, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl.

In certain embodiments, -A-B- is —CH═CH—CH═CH—.

In certain embodiments of compounds of Formula II, III, IV, V, VI, and VII, n is 0. In certain embodiments of compounds of Formula II, III, IV, V, VI, and VII, n is 1.

In certain embodiments, the compound of Formula I is chosen from
5-(5-phenyloxazol-2-yl)benzo[d]thiazole;
(1H-indol-3-yl)(5-phenyloxazol-2-yl)methanone;
1-(4-(5-phenyloxazol-2-yl)phenyl)-1H-pyrazole;
2-((4-methoxyphenoxy)methyl)-5-phenyloxazole;
2-(2-chlorophenyl)-5-phenyloxazole;
2-(2-phenyloxazol-5-yl)quinoline;
2-(3-chlorophenyl)-5-phenyloxazole;
2-(3-methoxyphenyl)-5-phenyloxazole;
2-(4-(morpholinylsulfonyl)phenyl)-5-phenyloxazole;
2-(4-(N,N-di-npropylsulfonyl)phenyl)-5-phenyloxazole;
2-(4-chlorophenyl)-5-phenyloxazole;
2-(4-methoxybenzyl)-5-phenyloxazole;
2-(4-methoxyphenyl)-5-phenyloxazole;
2-(4-tert-butylphenyl)-5-phenyloxazole;
2-(5-phenyloxazol-2-yl)quinoline;
2-(naphthalen-2-yl)-5-phenyloxazole;
2,5-diphenyloxazole;
2-chloro-4-(5-phenyloxazol-2-yl)pyridine;
2-chloro-5-(5-phenyloxazol-2-yl)pyridine;
2-phenoxy-5-(5-phenyloxazol-2-yl)pyridine;
3-((5-phenyloxazol-2-yl)methyl)pyridine;
3-((E)-2-(5-phenyloxazol-2-yl)vinyl)pyridine;
3-(2-(2-methoxyphenyl)oxazol-5-yl)pyridine;
3-(2-(3-methoxyphenyl)oxazol-5-yl)pyridine;
3-(2-(4-methoxyphenyl)oxazol-5-yl)pyridine;
3-(2-phenyloxazol-5-yl)pyridine;
3-(4-(5-phenyloxazol-2-yl)thiazol-2-yl)pyridine;
3-(5-(4-methoxyphenyl)oxazol-2-yl)pyridine;
3-(5-phenyloxazol-2-yl)-2H-chromen-2-one;
3-(5-phenyloxazol-2-yl)benzonitrile;
3-(5-phenyloxazol-2-yl)H-pyrazolo[1,5-a]pyridine;
3-(5-phenyloxazol-2-yl)pyridine;
4-((5-phenyloxazol-2-yl)methyl)pyridine;
4-(3-(5-phenyloxazol-2-yl)pyridin-2-yl)morpholine;
4-(5-(4-bromophenyl)oxazol-2-yl)pyridine;
4-(5-(4-iodophenyl)oxazol-2-yl)pyridine;
4-(5-(4-methoxyphenyl)oxazol-2-yl)benzoic acid;
4-(5-(4-methoxyphenyl)oxazol-2-yl)quinoline;
4-(5-(5-(pyridin-3-yl)oxazol-2-yl)pyridin-2-yl)morpholine;
4-(5-(5-phenyloxazol-2-yl)pyridin-2-yl)morpholine;
4-(5-phenyloxazol-2-yl)benzonitrile;
4-(5-phenyloxazol-2-yl)phenyl acetate;
4-(5-phenyloxazol-2-yl)pyridazine;
4-(5-phenyloxazol-2-yl)quinoline;
5-(4-bromophenyl)-2-(thiophen-2-yl)oxazole;
5-(5-phenyloxazol-2-yl)-1H-benzo[d][1,2,3]triazole;
5-(5-phenyloxazol-2-yl)-1H-benzo[d]imidazol-2(3H)-one;
5-(5-phenyloxazol-2-yl)-1H-benzo[d]imidazole;
5-(5-phenyloxazol-2-yl)isoquinoline;
5-(5-phenyloxazol-2-yl)pyrimidin-4-amine;
5-(5-phenyloxazol-2-yl)quinoline;
5-phenyl-2-(thiophen-2-yl)oxazole;
5-phenyl-2-m-tolyloxazole;
5-phenyl-2-o-tolyloxazole;
5-phenyl-2-p-tolyloxazole;
5-phenyl-2-styryloxazole;
6-(5-(4-chlorophenyl)oxazol-2-yl)-2-methyl-1H-benzo[d]imidazole;
6-(5-(4-methoxyphenyl)oxazol-2-yl)-2-methyl-1H-benzo[d]imidazole;
6-(5-(pyridin-3-yl)oxazol-2-yl)quinoxaline;
6-(5-phenyloxazol-2-yl)benzo[d]thiazole;
6-chloro-2-(5-phenyloxazol-2-yl)imidazo[1,2-b]pyridazine;
methyl 4-(5-(pyridin-3-yl)oxazol-2-yl)benzoate;
methyl 4-(5-phenyloxazol-2-yl)benzoate;
N-((5-phenyloxazol-2-yl)methyl)nicotinamide;
N-(4-(5-(pyridin-3-yl)oxazol-2-yl)phenyl)acetamide;
N-(4-(5-phenyloxazol-2-yl)pyridin-2-yl)acetamide;
N-(6-(5-phenyloxazol-2-yl)benzo[d]thiazol-2-yl)acetamide;
N,N-dimethyl-4-((E)-2-(5-phenyloxazol-2-yl)vinyl)benzenamine; and
N,N-dimethyl-4-(5-phenyloxazol-2-yl)benzenamine.

In certain embodiments, the methods described herein comprise administering at least one chemical entity chosen from
2-(benzo[d][1,3]dioxol-6-yl)-5-(2-fluorophenyl)-1,3,4-oxadiazole;
2-(4-(benzyloxy)-3-methoxyphenyl)-5-styryl-1,3,4-oxadiazole;
2-(benzo[d][1,3]dioxol-6-yl)-5-(furan-2-yl)-1,3,4-oxadiazole;
2-(4-ethoxyphenyl)-5-(4-fluorophenyl)-1,3,4-oxadiazole;
4-(4-(5-(4-fluorophenyl)-1,3,4-oxadiazol-2-yl)phenyl)morpholine;
4-(5-(4-chlorophenyl)-1,3,4-oxadiazol-2-yl)-N,N-dimethylbenzenamine;
4-(4-(5-(4-methoxyphenyl)-1,3,4-oxadiazol-2-yl)phenyl)morpholine;
3-(5-(3-aminophenyl)-1,3,4-oxadiazol-2-yl)benzenamine;
4-(5-(4-aminophenyl)-1,3,4-oxadiazol-2-yl)benzenamine;
4-(5-(3-methoxyphenyl)-1,3,4-oxadiazol-2-yl)benzenamine;
4-(5-(4-(difluoromethylsulfonyl)phenyl)-1,3,4-oxadiazol-2-yl)-N,N-dimethylbenzenamine;
4-(5-(4-ethoxyphenyl)-1,3,4-oxadiazol-2-yl)pyridine;
N,N-dimethyl-4-(5-(pyridin-3-yl)-1,3,4-oxadiazol-2-yl)benzenamine;
4-(5-(4-butoxyphenyl)-1,3,4-oxadiazol-2-yl)pyridine;
4-(5-(4-isobutoxyphenyl)-1,3,4-oxadiazol-2-yl)pyridine;
4-(5-(3,4-dimethoxyphenyl)-1,3,4-oxadiazol-2-yl)pyridine;
4-(5-(2,4-dichlorophenyl)-1,3,4-oxadiazol-2-yl)pyridine;
4-(5-(pyridin-4-yl)-1,3,4-oxadiazol-2-yl)benzenamine;
4-(5-p-tolyl-1,3,4-oxadiazol-2-yl)pyridine;
4-(5-(2-chlorophenyl)-1,3,4-oxadiazol-2-yl)pyridine;
4-(5-(pyridin-4-yl)-1,3,4-oxadiazol-2-yl)pyridine;
4-(5-phenyl-1,3,4-oxadiazol-2-yl)benzoic acid;
2-(5-phenyl-1,3,4-oxadiazol-2-yl)benzenamine;
2,5-diphenyl-1,3,4-oxadiazole;
3-(5-(2-bromophenyl)-1,3,4-oxadiazol-2-yl)-7-(diethylamino)-2H-chromen-2-one;
3-(5-(2-bromophenyl)-1,3,4-oxadiazol-2-yl)-6-methoxy-2H-chromen-2-one;
3-(5-(furan-2-yl)-1,3,4-oxadiazol-2-yl)-1-methyl-1H-indole;
1-methyl-3-(5-(thiophen-2-yl)-1,3,4-oxadiazol-2-yl)-1H-indole;
2-(3,4,5-trimethoxyphenyl)-5-(5-methylfuran-2-yl)-1,3,4-oxadiazole;
2-(4-methoxyphenyl)-5-(5-methylfuran-2-yl)-1,3,4-oxadiazole;
ethyl 2-(4-(5-(thiophen-2-yl)-1,3,4-oxadiazol-2-yl)phenoxy)acetate; and
3-(4-(5-(furan-2-yl)-1,3,4-oxadiazol-2-yl)phenylcarbamoyl)propanoic acid;
   and pharmaceutically acceptable salts, solvates, chelates, non-covalent complexes, prodrugs, and mixtures thereof.

When the chemical structure and chemical name conflict, the chemical structure is determinative of the identity of the compound. The chemical entities of the present disclosure may contain one or more chiral centers and/or double bonds and therefore, may exist as stereoisomers, such as double-bond isomers (i.e., geometric isomers), enantiomers or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into the component enantiomers or stereoisomers using separation techniques or chiral synthesis techniques well known to the skilled artisan.

Compounds of Formula I include, but are not limited to optical isomers of compounds of Formula I, racemates, and other mixtures thereof. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds of Formula I include Z- and E-forms (or cis- and trans-forms) of compounds with double bonds. Where compounds of Formula I exists in various tautomeric forms, chemical entities of the present disclosure include all tautomeric forms of the compound.

Chemical entities of the present disclosure include, but are not limited to compounds of Formula 1 and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. Hence, the terms “chemical entity” and “chemical entities” also encompass pharmaceutically acceptable salts, solvates, chelates, non-covalent complexes, prodrugs, and mixtures.

As used herein, the chemical entities of the present disclosure can include pharmaceutically acceptable derivatives or prodrugs thereof. A “pharmaceutically acceptable derivative or prodrug” refers to any appropriate pharmaceutically acceptable salt, ester, salt of an ester, hydrate, solvate, or other derivative of a compound of this present disclosure that, upon administration to a subject, is capable of providing, directly or indirectly, a compound of the present disclosure. Particularly favored derivatives and prodrugs include those that increase the bioavailability of the chemical entities of the present disclosure when such chemical entities are administered to a subject, for example by allowing an orally administered compound to be more readily absorbed into the blood, or which enhance delivery of the parent compound to a biological compartment, such as the brain or lymphatic system, relative to the parent species. Prodrugs can include derivatives where a group which enhances aqueous solubility or active transport through the gut membrane is appended to the structure of Formulae (I)-(V). Other prodrugs can include a promoiety that modifies the ADME (absorption, distribution, metabolism and excretion) of the parent compound and thereby enhances the therapeutic effectiveness of the parent compound.

In certain embodiments, chemical entities of the present disclosure can be modified by appending appropriate functionalities to enhance selective biological properties. Such modifications are known in the art and include those which can increase biological penetration into a given biological compartment, such as blood, lymphatic system, central nervous system, to increase oral availability, increase solubility to allow administration by injection, alter metabolism, and alter the rate of excretion.

In some embodiments, chemical entities of the present disclosure can be modified to facilitate use in biological assay, screening, and analysis protocols. Such modifications can include, for example, derivatizing to effect or enhance binding to physical surfaces such as beads or arrays, or modifying to facilitate detection such as by radiolabeling, affinity labeling, or fluorescence labeling.

Chemical entities of the present disclosure possess inhibitory activity with at least one ATP-utilizing enzyme. An ATP-utilizing enzyme refers to an enzyme that catalyzes the transfer of a phosphate group from an ATP molecule to a biomolecule such as a protein or carbohydrate. Examples of ATP-utilizing enzymes include, but are not limited to, synthetases, ligases, and kinases. The kinases can be animal kinases, including mammalian protein kinases, and human protein kinases.

Without being limited by theory, ATP-utilizing enzymes can be inhibited by compounds structurally similar to the phosphoryl-containing compounds that serve as the substrate for the phosphorylation reaction. For example, structurally similar compounds can bind to the active site or catalytic domain of an ATP-utilizing enzyme and thereby prevent substrate binding.

In certain embodiments, chemical entities of the present disclosure exhibited human protein kinase inhibitory activity.

Protein kinases are among the largest and most functionally diverse gene families. Most of the over 500 human protein kinases belong to a single superfamily of enzymes in which the catalytic domains are related in sequence and structure. Most human protein kinases can further be grouped into seven major groups based on the deoxyribonucleic acid (DNA) sequence homologies identified as CAMK (calcium/calmodulin-dependent protein kinases), AGC (including PKA (protein kinase A), PKG (protein kinase G), PKC (protein kinase C) kinases), CK1 (casein kinases), CMGC (containing CDK (cyclin-dependent), MAPK (mitogen activated), GSK3 (glycogen synthase) and CLK (CDC2-like) kinases), STE (homologs of yeast Sterile 7, Sterile 11, and Sterile 20 kinases), TK (tyrosine kinases), and TKL (tyrosine-kinase like).

The AGC protein kinase family includes AKT1, AKT2, AKT3, AURORA-A, MSK1, MSK2, P70S6K, PAK1, PKA, and SGK1 protein kinases. The CMGC protein kinase family includes the CDK1, CDK2/cyclinA, CDK2/cyclinE, CDK5, DYRK2, GSK3-α, GSK3-β, P38-α, P38-β, P38-δ, and P38-γ, and MAPK1 protein kinases. The CAMK protein kinase family includes the DAPK1, MAPKAPK2, CHEK1, CHEK2, PRAK, and c-TAK1 protein kinases. The TK protein kinase family includes the ABL1, CSK, FLT3, FYN, HCK, INSR, KIT, LCK, PDGFR-α, LYNA, SYK, and SRC protein kinases. The STE protein kinase family includes PAK2 protein kinase.

Certain chemical entities of the present disclosure exhibited selectivity for one or more protein kinases, where selectivity is as defined herein. Certain chemical entities of the present disclosure exhibited selective activity for at least one of the following protein kinases: AURORA-A, CK2, FLT3, c-KIT, PDGFR-α, PDGFR-β, GSK3-α, PDK1 and c-TAK1. Certain chemical entities of the present disclosure exhibited selective activity for FLT3, c-KIT, PDGFR-α, or PDGFR-β.

Chemical entities of the present disclosure can be prepared by methods well known in the art.

Chemical entities of the present disclosure can be prepared from readily available starting materials using the flowing general methods and procedures. It will be appreciated that where typical or preferred process conditions, such as, reaction temperatures, times, mole ratios of reactants, solvents, pressures, are given, other process conditions can also be used unless otherwise stated. Reaction conditions may vary with the reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, 3^{rd} Edition, John Wiley & Sons, 1999, and references cited therein.

Furthermore, chemical entities of the present disclosure can contain one or more chiral centers. Accordingly, if desired, such compounds can be prepared or isolated as pure stereoisomers, i.e., as individual enantiomers or diastereomers, or as stereoisomer-enriched mixtures. All such stereoisomers, and enriched mixtures thereof, are included within the scope of the present disclosure, unless otherwise indicated. Pure stereoisomers, and enriched mixtures thereof, can be prepared using, for example, optically active starting materials or stereoselective reagents well-known in the art. Alternatively, racemic mixtures of such compounds can be separated using, for example, chiral column chromatography, chiral resolving agents and the like.

General synthetic schemes and specific reaction protocols used to prepare chemical entities of the present disclosure are presented in the reaction schemes and Examples provided herein.

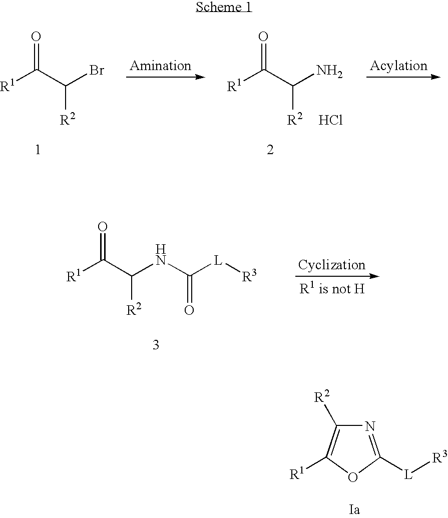

The substituted oxazole compounds Ia of the present invention are prepared by a variety of known procedures, such as those described in Science of Synthesis 2002, 11, 383-479, Journal of Organic Chemistry 2003, 68, 839-853, and US20010006976. For example, oxazole compounds of formula Ia (R¹ is not H) can be prepared via cyclization of intermediates 3, which in turn can be derived from compounds of formula 2 via acylation with the appropriate R³-acylating agent using known methods for the formation of an amide bond. Amination reaction of compounds 1 can afford compounds 2 (see Journal of Organic Chemistry 2003, 68, 839-853). Compounds of formula 1 are commercially available or can be prepared in accordance with known methods, such as those described in J. March, Advanced Organic Chemistry 4^{th} Ed., John Wiley & Sons, 1992, p. 587-590.

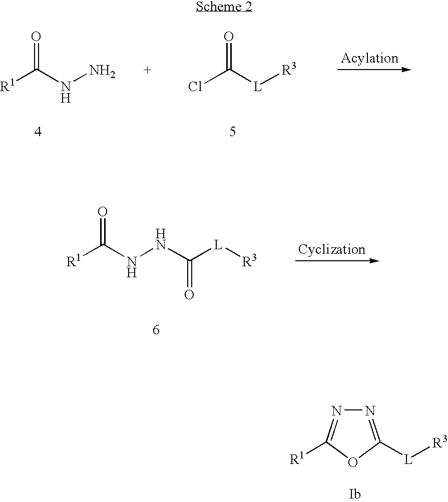

The substituted oxadiazole compounds Ib of the present invention are prepared by a variety of known procedures, such as those described in Synthetic Communications, 2004, 34, 2387-2391 and Organic Letters, 2005, 7, 1039 via cyclization of compounds of formula 6. Compounds of formula 4 and 5, which serve as starting materials are commercially available or can be prepared in accordance with methods known in the art.

In accordance with certain embodiments, chemical entities of the present disclosure exhibit ATP-utilizing enzyme inhibitory activity. Thus, one important use of the chemical entities of the present disclosure includes the administration of at least one chemical entity of the present disclosure to a subject, such as a human. This administration serves to arrest, ameliorate, reduce the risk of acquiring, reduce the development of or at least one of the clinical symptoms of, or reduce the risk of developing or at least one of the clinical symptoms of diseases or conditions regulated by ATP-utilizing enzymes, such as, protein kinases.

For example, unregulated or inappropriately high protein kinase activity has been implicated in many diseases resulting from abnormal cellular function. Unregulated or inappropriately high protein kinase activity can arise either directly or indirectly, for example, by failure of the proper control mechanisms of a protein kinase, related, for example, to mutation, over-expression or inappropriate activation of the enzyme; or by over- or under-production of cytokines or growth factors also participating in the transduction of signal upstream or downstream of the protein kinase. In all of these instances, selective inhibition of the action of a protein kinase can be expected to have a beneficial effect.

According to certain embodiments, the present disclosure relates to methods of treating a disease regulated by at least one ATP-utilizing enzyme in a subject. ATP-utilizing enzyme regulated diseases include, for example, those where the ATP-utilizing enzyme participates in the signaling, mediation, modulation, control or otherwise involved in the biochemical processes affecting the manifestation of a disease. In certain embodiments, the methods are useful in treating diseases regulated by protein kinase enzymes. Protein kinase regulated diseases include, for example, the following general disease classes: cancer, autoimmunological, metabolic, inflammatory, infection, diseases of the central nervous system, degenerative neural disease, allergy/asthma, angiogenesis, neovascularization, vasculogenesis, cardiovascular, and the like. Without being limited by theory, specific examples of diseases that are known or believed to be regulated by protein kinase enzymes, include, transplant rejection, osteoarthritis, rheumatoid arthritis, multiple sclerosis, diabetes, diabetic retinopathy, asthma, inflammatory bowel disease such as Crohn's disease, and ulcerative colitis, renal disease cachexia, septic shock, lupus, diabetes mellitus, myasthenia gravis, psoriasis, dermatitis, eczema, seborrhea, Alzheimer's disease, Parkinson's disease, stem cell protection during chemotherapy, ex vivo selection or ex vivo purging for autologous or allogeneic bone marrow transplantation, leukemia including, but not limited to, acute myeloid leukemia, chronic myeloid leukemia, and acute lymphoblastic leukemia, cancer including but not limited to, breast cancer, lung cancer, colorectal cancer, ovary cancer, prostate cancer, renal cancer, squamous cell cancer, glioblastoma, melanoma, pancreatic cancer, and Kaposi's sarcoma, ocular disease, corneal disease, glaucoma, bacterial infections, viral infections, fungal infections, heart disease, stroke, obesity, endometriosis, atherosclerosis, vein graft stenosis, peri-anastomatic prosthetic graft stenosis, prostate hyperplasia, chronic obstructive pulmonary disease, inhibition of neurological damage due to tissue repair, scar tissue formation, wound healing, pulmonary disease, neoplasm, macular degeneration.

Chemical entities of the present disclosure are particularly useful for the treatment of cancer including, but are not limited to, glioblastoma, ovarian cancer, breast cancer, endometrial carcinoma, hepatocellular carcinoma, melanoma, colorectal cancer, colon cancer, digestive tract, lung cancer, renal-cell carcinoma, thyroid, lymphoid, prostate cancer and pancreatic cancer, etc. advanced tumors, hairy cell leukemia, melanoma, chronic myelogenous leukemia, advanced bead and neck. metastatic renal cell, non-Hodgkin's lymphoma, metastatic breast, breast adenocarcinoma. advanced melanoma. pancreatic, gastric, non-small cell lung, small cell lung, renal cell carcinoma. various solid tumors, multiple myeloma, metastatic prostate, malignant glioma. renal cancer, lymphoma. refractory metastatic disease, refractory multiple myeloma, cervical cancer, Kaposi's sarcoma, recurrent anaplastic glioma. and metastatic colon cancer.

More particularly, cancers that may be treated by chemical entities of the present disclosure, include, but are not limited to: Cardiac: sarcoma (angiosarcoma, fibrosarcoma, rhabdomyosarcoma, liposarcoma), myxoma, rhabdomyoma, fibroma, lipoma, teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous, cell carcinoma, adenocarcinoma, leiomyosarcoma, lymphoma) stomach (carcinoma, lymphoma, leiomyosarcoma), pancreas (ductal adenocarcinoma, insulinoma, glucagonoma, gastrinoma, carcinoid tumors, Karposi's sarcoma, leiomyoma, hemangioma, lipoma, neurofibroma, fibroma), large bowel (adenocarcinomas, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chrondroma, chondroblastoma, chondromyxofibroma, osteoid osteoma and giant cell tumors; Nervous system: skull (osteoma, hemangioma, granuloma, xanthoma, osteitis deformans, meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord, neurofibroma, meningioma, glioma, sarcoma); Gynecological: uterus (endometrial carcinoma), cervix (cervical carcinoma, pre-tumor cervical displasia), ovaries (ovarian carcinoma [serous cystadenocarcinoma, mucinous cystadenocarcinoma], granulose-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma) vagina (clear cell carcinoma, squamous cell carcinoma, botryoid sarcoma (embryonal rhabdomyosarcoma), fallopian tubes carcinoma); Hematologic: blood (myeloid leukemia (acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma.

Chemical entities of the present disclosure may also be useful for the treatment of tuberous sclerosis complex.

Chemical entities of the present disclosure may also be useful for the treatment of other conditions (e.g., inflammatory disease), including, but are not limited to, rheumatoid arthritis, osteoarthritis, endometriosis, atherosclerosis, vein graft stenosis, peri-anastomatic prosthetic graft stenosis, prostate hyperplasia, chronic obstructive pulmonary disease, psoriasis, inhibition of neurological damage due to tissue repair, scar tissue formation, wound healing, multiple sclerosis, inflammatory bowel disease, infections, particularly bacterial, viral, retroviral or parasitic infections (by increasing apoptosis), pulmonary disease, neoplasm, Parkinson's disease, transplant rejection (as an immunosuppressant), macular degeneration and septic shock.

Chemical entities of the present disclosure may also be useful for the treatment of diseases mediated by, but not limited to, modulation or regulation of KIT, FLT, or PDGFR protein kinases, additional tyrosine kinases, serine/threonine kinases, and/or dual specificity kinases.

In certain embodiments, a pharmaceutical composition can include at least one chemical entity of the present disclosure and at least one additional therapeutic agent appropriate for effecting combination therapy. Chemical entities of the present disclosure are also useful in combination with known therapeutic agents and anti-cancer agents. A person skilled in the art would be able to discern which combinations of agents would be useful based on the particular characteristics of the drugs and the cancer involved. Many chemotherapeutics are presently known in the art. Many chemotherapeutics are presently known in the art. Such anti-cancer agents include, but are not limited to, estrogen receptor modulators, cytostatic/cytotoxic agents, anti-proliferative agents, cell cycle checkpoint inhibitors, angiogenesis inhibitors, monoclonal antibody targeted therapeutic agents, tyrosine kinase inhibitors, serine-threonine kinase inhibitors, histone deacetylase inhibitors, heat shock protein inhibitors, and farnesyl transferase inhibitors. Chemical entities of the present disclosure are also useful in combination with radiation therapy.

Examples of cytostatic/cytotoxic agents, anti-proliferative agents and cell cycle checkpoint inhibitors include, but are not limited to, sertenef, cachectin, ifosfamide, tasonermin, lonidamine, carboplatin, altretamine, prednimustine, dibro-modulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, porfiromycin, cisplatin, irofulven, dexifosfamide, cis-aminedichloro(2-methylpyridine)platinum, benzylguanine, glufosfamide, GPXlOO, (trans, trans, trans)-bis-mu-(hexane-l,6-diamine)-mu[di-amine-platinum(II)]bis[diamine(chloro)platinum (II)]tetrachloride, diarizidinylspermine, arsenic trioxide,l-(ll-dodecylamino-l0-hydroxyundecyl)-3,7-dimethylxanthine, zocubicin, idarubicin, daunorubicin, bisantrene, mitoxantrone, pirarubicin, pinafide, valrubicin, amrubicin, antineoplaston, 3′-diamino-3′-morpholino-13-deoxo-10-hydroxy-caminomycin, annamycin, galarubicin, elioafide, MENI0755, and 4-demethoxy-3-deamino-3-aziridinyl-4-methylsulphonyl-daunorubicin.

An example of a hypoxia activatable compound is tirapazamine.

Examples of proteosome inhibitors include but are not limited to lactacystin and MLN-341 (Velcade).

Examples of microtubule inhibitors/microtubule-stabilizing agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPRI09881, BMS184476, vinflunine, and BMS188797.

Some examples of topoisomerase inhibitors are topotecan, bycaptamine, irinotecan, robitecan, 6-ethoxypropionyl-3′,4′-O-exo-benzylidene-chartreusin.

“Inhibitors of kinases” involved in mitotic progression include, but are not limited to, inhibitors of aurora kinases, inhibitors of Polo-like kinases (PLK; in particular inhibitors of PLK-1), inhibitors of bub-1 and inhibitors of bub-R1.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine. trimetrexate, fludarabine, capecitabine, galocitabine, cytarabine ocfosfate, fosteabine sodium hydrate, raltitrexed, paltitrexid, emitefur, tiazofurin, decitabine, nolatrexed, pem-etrexed, nelzarabine.

Examples of monoclonal antibody targeted therapeutic agents include those therapeutic agents which have cytotoxic agents or radioisotopes attached to a cancer cell specific or target cell specific monoclonal antibody. Examples can be found in a number of references (Krause and Van Etten, 2005 New Eng. J. Med. 353, 172184) and include, but are not limited to, Bexxar, trastuzumab (herceptin), cetuximab (erbitux), ABX-EGF, 2C4, bevacizumab (avastin), bortezomib, rituxan.

Some specific examples of tyrosine inhibitors can be found in a number of references (Krause and Van Etten, 2005 New Eng. J. Med. 353, 172184; Brown and Small 2004 Eur. J. Cancer 40, 707-721; Fabian et al. 2005 Nat. Biotech. 23, 329-336) and include imatinib (Gleevec, STI571), gefitnib (Iressa), BMS-354825, PKC412, PD 0173074, SU5402, MLN-518, CEP-701, SU5416, erlotinib (tarceva), CI-1033, CT2923, sunitinib (SU11248), GW-2016, EKB-569, ZD-6474, vatalanib (PTK-787), AMN107, ZD6474, CHIR-258, OSI-930, AZD0530, AEE788.

Some specific examples of serine/threonine kinase inhibitors can be found in a number of references (Jackman et al. 2004 Drug Disc Today: Ther Strategies 1, 445-454; Fabian et al. 2005 Nat. Biotech. 23, 329-336; Pearson and Fabbro 2004, Expert Rev. Anticancer Ther. 4, 1113-1124) and include but are not limited to, LY-333531, sorafenib (BAY-43-9006), roscovitine (CYC202), CI-1040, ZM447439, CCI-779, RAD001, UNC01, VX680, AP23573.

Examples of heat shock protein inhibitors include, but are not limited to, 17-AAG and 17-DMAG.

Examples of histone deacetylase inhibitors include, but are not limited to, MS-275, AN-9, apicidin derivatives, Baceca, CBHA, CHAPs, chlamydocin, CS-00028, CS-055, EHT-0205, FK-228, FR-135313, G2M-777, HDAC-42, LBH-589, MGCD-0103, NSC-3852, PXD-101, pyroxamide, SAHA derivatives, suberanilohydroxamic acid, tacedinaline, VX-563, and zebularine.

Examples of farnesyl transferase inhibitors include, but are not limited to, lonafarnib.

Certain embodiments of the present disclosure are directed to methods of treating disease in a subject comprising the step of administering to a subject, in need of such treatment, a therapeutically effective amount of at least one chemical entity of the present disclosure. In some embodiments, a disease can be regulated by at least one ATP-utilizing enzyme such as a protein kinase. Certain diseases can be regulated by one or more ATP-utilizing enzymes. In such cases, treatment of the disease or disorder can include administering a therapeutically effective amount of at least one chemical entity of the present disclosure that inhibits the activity of one or more ATP-utilizing enzymes, or more than one compound of the present disclosure, wherein each compound inhibits at least one different ATP-utilizing enzyme.

Other embodiments of the present disclosure are related to methods of inhibiting at least one ATP-utilizing enzyme, including for example, a protein kinase. In certain embodiments, the ATP-utilizing enzyme can be inhibited by the method of administering to a subject, at least one chemical entity of the present disclosure, or a composition comprising at least one chemical entity of the present disclosure.

In certain embodiments, the present disclosure relates to methods of inhibiting ATP-utilizing enzyme activity by contacting at least one ATP-utilizing enzyme with at least one chemical entity of the present disclosure. ATP-utilizing enzymes include phosphotransferase enzymes that catalyze the phosphorylation of a biological molecule by transferring a phosphate group from an ATP substrate. ATP-utilizing enzymes include for example, synthetases, ligases, and kinases. Certain methods of the present disclosure are useful in inhibiting protein kinase enzymes, including, for example, the following protein kinase enzymes: AURORA-A, CK2, FLT3, c-KIT, PDGFR-α, PDGFR-β, GSK3-α, PDK1 and c-TAK1.

In certain embodiments, at least one of the protein kinases is a tyrosine receptor kinase, including but not limited to wild-type and mutant PDGFR-α, PDGFR-β, FLT-3, and c-KIT receptors. In certain embodiments, at least one of the protein kinases is FLT3. In certain embodiments, at least one of the protein kinases is c-KIT. In certain embodiments, at least one of the protein kinases is PDGFR-α or PDGFR-β.

Some methods of the present disclosure can be used to inhibit ATP-utilizing enzymes that are present in a living organism, such as a mammal; contained in a biological sample such as a cell, cell culture, or extract thereof, biopsied material obtained from a mammal or extracts thereof, and blood, saliva, feces, semen, tears or other body fluids or extracts thereof; contained within a reagent, or bound to a physical support. In certain embodiments, an ATP-utilizing enzyme can regulate a disease or disorder and in other embodiments, the ATP-utilizing enzyme may not regulate a disease or disorder.

According to the methods of the present disclosure, at least one ATP-utilizing enzyme can be inhibited by contact with at least one chemical entity of the present disclosure. In vivo ATP-utilizing enzymes can be inhibited by administration through routes and using compositions comprising at least one chemical entity of the present disclosure. For in vitro systems, contacting an ATP-utilizing enzyme with at least one chemical entity of the present disclosure can include, for example, combining liquid reagents or combining a reagent and an ATP-utilizing enzyme and/or compound of the present disclosure attached to a solid support. The ATP-utilizing enzyme and compound of the present disclosure can be contacted in any appropriate device such as an affinity chromatography column, a microarray, a microfluidic device, assay plate, or other appropriate chemical or biotechnology apparatus used to perform biochemical analysis, assay, screening, and the like.

In certain embodiments, pharmaceutical compositions of the present disclosure may be administered orally, parenterally, by inhalation spray, topically, rectally, nasally, buccally, vaginally, via an implanted reservoir, or by any other appropriate route. Pharmaceutical compositions of the present disclosure can contain one or more pharmaceutically acceptable vehicles. In some embodiments, the pH of the formulation can be adjusted with pharmaceutically acceptable acids, bases or buffers to enhance the stability of the formulated compound or the delivery form. The term parenteral as used herein includes subcutaneous, intracutaneous, intravenous, intramuscular, intra-articular, intra-arterial, intrasynovial, intrasternal, intrathecal, intralesional, and intracranial injection or infusion techniques.

In certain embodiments, compounds disclosed herein can be delivered orally. Suitable dosage ranges for oral administration can depend on the potency of the compounds, but generally can range from 0.1 mg to 20 mg of a compound per kilogram of body weight. Appropriate dosages can be in the range of 25 to 500 mg/day and the dose of compounds administered can be adjusted to provide an equivalent molar quantity of compound in the plasma of a subject. Dosage ranges can be readily determined by methods known to those skilled in the art.

A dosage can be delivered in a composition by a single administration, by multiple applications, by sustained release or by controlled sustained release, or any other appropriate intervals and/or rates of release.

Chemical entities of the present disclosure can be assayed in vitro and in vivo, for the desired therapeutic or prophylactic activity prior to therapeutic use in mammals. For example, in vitro assays can be used to determine whether administration of a specific compound of the present disclosure or a combination of such compounds is effective for inhibiting the activity of certain ATP-utilizing enzymes or treating at least one disease. Chemical entities of the present disclosure can also be demonstrated to be effective and safe using animal model systems. A therapeutically effective dose of at least one chemical entity of the present disclosure can, in certain embodiments, provide therapeutic benefit without causing substantial toxicity. Toxicity of chemical entities of the present disclosure can be determined using standard pharmaceutical procedures and can be readily ascertained by the skilled artisan. The dose ratio between toxic and therapeutic effect is the therapeutic index. Chemical entities of the present disclosure can exhibit high therapeutic indices in treating diseases and disorders. The dosage of a compound of the present disclosure can be within a range of circulating concentrations that include an effective dose with little or no toxicity.

When employed as pharmaceuticals, chemical entities of the present disclosure can be administered in the form of pharmaceutical compositions. Such compositions can be prepared in a manner well known in the pharmaceutical art and can comprise at least one chemical entity of the present disclosure.

Pharmaceutical compositions of the present disclosure can comprise a therapeutically effective amount of at least one chemical entity of the present disclosure, and at least one pharmaceutically acceptable vehicle. Pharmaceutical compositions of the present disclosure can additionally comprise at least additional compound that enhances the therapeutic efficacy of one or more chemical entities of the present disclosure. For example, such compounds can enhance the therapeutic efficacy of chemical entities of the present disclosure by effectively increasing the plasma concentration of the compounds. Without being limited by theory, certain compound can decrease the degradation of the chemical entities of the present disclosure prior to administration or during transport to the plasma, or within the plasma. Certain compounds can increase the plasma concentration by increasing the absorption of compounds in the gastrointestinal tract. Pharmaceutical compositions of the present disclosure can also include additional therapeutic agents that are normally administered to treat a disease or disorder.

In certain embodiments, a pharmaceutical composition can include at least one chemical entity of the present disclosure and at least one additional therapeutic agent appropriate for effecting combination therapy.

In some embodiments, chemical entities and compositions of the present disclosure can be administered by oral routes. The compositions can be prepared in a manner well known in the pharmaceutical art and can comprise at least one chemical entity of the present disclosure. In some embodiments, compositions of the present disclosure contain a therapeutically effective amount of at least one chemical entity of the present disclosure, which can be in purified form, together with a therapeutically effective amount of at least one additional therapeutic agent, and a suitable amount of at least one pharmaceutically acceptable excipient, so as to provide the form for proper administration to a subject

Some embodiments of the present disclosure are directed to compositions that contain, as the active ingredient, of one or more chemical entities of the present disclosure associated with pharmaceutically acceptable excipients. In making certain compositions of the present disclosure, the active ingredient can be mixed with an excipient, diluted by an excipient, or enclosed within such a carrier that can be in the form of a capsule, sachet, paper or other container. When the excipient serves as a diluent, the excipient can be a solid, semi-solid, or liquid material, which acts as a vehicle, carrier or medium for the active ingredient. Thus, for example, the compositions can be in the form of tablets, pills, powders, lozenges, sachets, cachets, elixirs, suspensions, emulsions, solutions, and syrups containing, for example, from 1% to 90% by weight of at least one chemical entities of the present disclosure using, for example, soft and hard gelatin capsules.

In preparing a composition, it can be necessary to mill the active compound to provide the appropriate particle size prior to combining with other ingredients. If the active compound is insoluble, the active component ordinarily can be milled to a particle size of less than 200 mesh. If the active compound is water soluble, the particle size can be adjusted by milling to provide a uniform distribution in the formulation, e.g. 40 mesh.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, modified cyclodextrins, cellulose, water, syrup, and methyl cellulose. Some compositions can additionally include, lubricating agents such as talc, magnesium stearate, and mineral oil, wetting agents, emulsifying and suspending agents, preserving agents such as methyl- and propylhydroxy-benzoates, sweetening agents, and flavoring agents. Compositions of the present disclosure can be formulated so as to provide quick, sustained or delayed release of the active ingredient after administration to the subject by employing procedures known in the art.

Some compositions of the present disclosure can be formulated in unit dosage form, each dosage containing, for example, 0.1 mg to 2 g of the active ingredient. As used herein, “unit dosage forms” refers to physically discrete units suitable as unitary dosages for human subjects and other mammals, each unit containing a predetermined quantity of active material calculated to produce the desired therapeutic effect, in association with a suitable pharmaceutical excipient, diluent, carrier and/or adjuvant. In certain embodiments, compositions of the present disclosure can be formulated in multiple dosage forms. The amount of the chemical entities of the present disclosure that can be combined with other materials and therapeutic agents to produce compositions of the present disclosure in a single dosage form will vary depending upon the subject and the particular mode of administration.

In the treatment of disease, chemical entities of the present disclosure can be administered in a therapeutically effective amount. It will be understood, however, that the amount of the compound administered will be determined by a physician, in the light of the relevant circumstances, including the condition to be treated, the chosen route of administration, the actual compound administered, the age, weight, and response of the individual subject, the severity of the subject's symptoms, and the like.

For preparing solid compositions such as tablets, the principal active ingredient can be mixed with a pharmaceutical excipient to form a solid preformulation composition containing a homogeneous mixture of a compound of the present disclosure. When referring to these preformulation compositions as homogeneous, it is meant that the active ingredient is dispersed evenly throughout the composition so that the composition may be readily subdivided into equally effective unit dosage forms such as tablets, pills and capsules. The solid preformulation can then subdivided into unit dosage forms of the type described above containing from, for example, 0.1 mg to 2 g of the therapeutically effective compound of the present disclosure.

The tablets or pills comprising certain compositions of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials include a number of polymeric acids and mixtures of polymeric acids with such materials as shellac, cetyl alcohol, and cellulose acetate.

The liquid forms in which the compositions of the present disclosure may be incorporated for administration orally or by injection include aqueous solutions suitably flavored syrups, aqueous or oil suspensions, and flavored emulsions with edible oils such as cottonseed oil, sesame oil, coconut oil, or peanut oil, as well as elixirs and similar pharmaceutical vehicles.

As used herein, a “pharmaceutically acceptable derivative or prodrug” refers to any pharmaceutically acceptable salt, ester, salt of an ester or other derivative of a compound of the present disclosure that, upon administration to a recipient, is capable of providing, either directly or indirectly, a compound of the present disclosure or an inhibitory active metabolite or residue thereof. Examples of such derivates or prodrugs include those that increase the bioavailability of the chemical entities of the present disclosure when such compounds are administered to a mammal, e.g., by allowing an orally administered compound to be more readily absorbed into the blood, or which enhance delivery of the parent compound to a biological compartment, e.g., the brain or lymphatic system, relative to the parent species.

In certain embodiments, acceptable formulation materials can be nontoxic to recipients at the dosages and concentrations employed.

In certain embodiments, a pharmaceutical composition of the present disclosure can contain formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids such as glycine, glutamine, asparagine, arginine or lysine; antimicrobials; antioxidants such as ascorbic acid, sodium sulfite, or sodium hydrogen-sulfite; buffers such as borate, bicarbonate, Tris-HCl, citrates, phosphates or other organic acids; bulking agents such as mannitol or glycine; chelating agents such as ethylenediamine tetraacetic acid (EDTA); complexing agents such as caffeine, polyvinylpyrrolidone, beta-cyclodextrin, hydroxypropyl-beta-cyclodextrin, or sulfobutyl ether β-cyclodextrin; fillers; monosaccharides; disaccharides; and other carbohydrates such as glucose, mannose, or dextrins; proteins such as serum albumin, gelatin or immunoglobulins; coloring, flavoring and diluting agents; emulsifying agents; hydrophilic polymers such as polyvinylpyrrolidone; low molecular weight polypeptides; salt-forming counterions such as sodium; preservatives such as benzalkonium chloride, benzoic acid, salicylic acid, thimerosal, phenethyl alcohol, methylparaben, propylparaben, chlorhexidine, sorbic acid or hydrogen peroxide; solvents such as glycerin, propylene glycol or polyethylene glycol; sugar alcohols such as mannitol or sorbitol; suspending agents; surfactants or wetting agents such as pluronics, PEG, sorbitan esters, polysorbates such as polysorbate 20, polysorbate 80, triton, tromethamine, lecithin, cholesterol, tyloxapol; stability enhancing agents such as sucrose or sorbitol; tonicity enhancing agents such as alkali metal halides, such as sodium or potassium chloride, mannitol, sorbitol; delivery vehicles; diluents; excipients and/or pharmaceutical adjuvants (Remington's Pharmaceutical Sciences, 18^{th} Edition, A. R. Gennaro, ed., Mack Publishing Company (1990)).

In certain embodiments, the optimal pharmaceutical composition can be determined by one skilled in the art depending upon, for example the intended route of administration, delivery format, and desired dosage. See, for example, Remington's Pharmaceutical Sciences, supra. In certain embodiments, such compositions may influence the physical state, stability, rate of in vivo release, and rate of in vivo clearance of the antibodies of the present disclosure.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, neutral buffered saline or saline mixed with serum albumin are further exemplary vehicles. In certain embodiments, pharmaceutical compositions comprise Tris buffer of pH 7 to 8.5, or acetate buffer of pH 4 to 5.5, which can further comprise sorbitol or a suitable substitute thereof. In certain embodiments, buffers are used to maintain the composition at physiological pH or at a slightly lower pH, typically within a pH range of from 5 to 8.

In certain embodiments, pharmaceutical compositions of the present disclosure can be selected for parenteral delivery. In other embodiments, compositions can be selected for inhalation or for delivery through the digestive tract, such as orally. The preparation of such pharmaceutically acceptable compositions is within the skill of the art.

In certain embodiments, composition components can be present in concentrations that are acceptable to the site of administration. In certain embodiments, when parenteral administration is contemplated, a therapeutic composition can be in the form of a pyrogen-free, parenterally acceptable aqueous solution comprising at least one chemical entity of the present disclosure, with or without additional therapeutic agents, in a pharmaceutically acceptable vehicle. In other embodiments, a vehicle for parenteral injection can be sterile distilled water in which at least one chemical entity of the present disclosure, with or without at least one additional therapeutic agent, is formulated as a sterile, isotonic solution, properly preserved. In still other embodiments, the pharmaceutical composition can include encapsulation of at least one chemical entity of the present disclosure with an agent, such as injectable microspheres, bio-erodible particles, polymeric compounds such as polyacetic acid or polyglycolic acid, beads or liposomes, that can provide the controlled or sustained release of the compound of the present disclosure which can then be delivered via a depot injection. In certain embodiments, implantable drug delivery devices can be used to introduce a compound of the present disclosure to the plasma of a subject, within a target organ, or to a specific site within the subject's body.

In certain embodiments, a pharmaceutical composition can be formulated for inhalation. In certain embodiments, a compound of the present disclosure, with or without at least one additional therapeutic agent, can be formulated as a dry powder for inhalation. In certain embodiments, an inhalation solution comprising a compound of the present disclosure with or without at least one additional therapeutic agent can be formulated with a propellant for aerosol delivery. In other embodiments, solutions can be nebulized. In still other embodiments, solutions, powders or dry films of chemical entities of the present disclosure can be aerosolized or vaporized for pulmonary delivery.

In certain embodiments, it is contemplated that formulations can be administered orally. In certain embodiments, a compound of the present disclosure, with or without at least one additional therapeutic agent that can be administered orally, can be formulated with or without carriers customarily used in the compounding of solid dosage forms such as tablets and capsules. In other embodiments, a capsule may be designed to release the active portion of the formulation in the region of the gastrointestinal tract where bioavailability can be maximized and pre-systemic degradation minimized. In still other embodiments, at least one additional agent can be included in the formulation to facilitate absorption of the compound of the present disclosure and/or any additional therapeutic agents into the systemic circulation. In certain embodiments, diluents, flavorings, low melting point waxes, vegetable oils, lubricants, suspending agents, tablet disintegrating agents, and binders can be employed.

In certain embodiments, a pharmaceutical composition of the present disclosure can include an effective quantity of chemical entities of the present disclosure, with or without at least one additional therapeutic agent, in a mixture with at least one pharmaceutically acceptable vehicle suitable for the manufacture of tablets. In certain embodiments, by dissolving the tablets in sterile water, or other appropriate vehicle, solutions can be prepared in unit-dose form. In certain embodiments, suitable excipients include inert diluents, such as calcium carbonate, sodium carbonate or bicarbonate, lactose, or calcium phosphate; or binding agents, such as starch, gelatin, or acacia; and lubricating agents such as magnesium stearate, stearic acid or talc.

In certain embodiments, the frequency of dosing will take into account the pharmacokinetic parameters of the chemical entities of the present disclosure and/or any additional therapeutic agents in the pharmaceutical composition used. In certain embodiments, a clinician can administer the composition until a dosage is reached that achieves the desired effect. The composition can be administered as a single dose, or as two or more doses, which may or may not contain the same amount of the therapeutically active compound time, or as a continuous infusion via an implantation device or catheter. Further refinement of an appropriate dosage can be routinely made by those of ordinary skill in the art. For example, therapeutically effective amounts and regimens can be determined through use of appropriate dose-response data.

In certain embodiments, the route of administration of the pharmaceutical composition can be in accord with known methods, e.g. orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions can be administered by bolus injection or continuously by infusion, or by an implantation device.

In certain embodiments, the composition can be administered locally via implantation of a membrane, sponge or another appropriate material onto which the desired compound of the present disclosure has been absorbed or encapsulated. In certain embodiments, where an implantation device is used, the device can be implanted into any suitable tissue or organ, and delivery of the desired molecule via diffusion, timed-release bolus, or continuous administration.

In certain embodiments, it can be desirable to use a pharmaceutical composition comprising a compound of the present disclosure, with or without at least one additional therapeutic agent, in an ex vivo manner. For example, cells, tissues and/or organs that have been removed from a subject are exposed to a pharmaceutical composition comprising a compound of the present disclosure, with or without at least one additional therapeutic agent, after which the cells, tissues and/or organs are subsequently implanted back into the subject.

Pharmaceutical compositions according to the present disclosure can take a form suitable for oral, buccal, parenteral, nasal, topical or rectal administration, or a form suitable for administration by inhalation or insufflation.

The compositions of the present disclosure can, if desired, be presented in a pack or dispenser device that can contain one or more unit dosage forms containing the active ingredient. The pack or dispensing device can be accompanied by instructions for administration.

The quantity of a compound of the present disclosure required for the treatment of a particular condition can vary depending on the compound, and the condition of the subject to be treated. In general, daily dosages can range from 100 ng/kg to 100 mg/kg, e.g., 0.01 mg/kg to 40 mg/kg body weight, for oral or buccal administration; from 10 ng/kg to 50 mg/kg body weight, e.g., 0.001 mg/kg to 20 mg/kg body weight, for parenteral administration; and from 0.05 mg to 1,000 mg for nasal administration or administration by inhalation or insufflation.

Certain chemical entities of the present disclosure and/or compositions of the present disclosure can be administered as sustained release systems. In certain embodiments, the chemical entities of the present disclosure can be delivered by oral sustained release administration. In this embodiment, the chemical entities of the present disclosure can be administered, for example, twice per day and, once per day.

The chemical entities of the present disclosure can be practiced with a number of different dosage forms, which can be adapted to provide sustained and/or extended release of a compound upon oral administration. Examples of sustained and/or extended release dosage forms include, but are not limited to, beads comprising a dissolution or diffusion release composition and/or structure, an oral sustained release pump, enteric-coated preparations, compound-releasing lipid matrices, compound releasing waxes, osmotic delivery systems, bioerodible polymer matrices, diffusible polymer matrices, a plurality of time-release pellets, and osmotic dosage forms.

Regardless of the specific form of sustained release oral dosage form used, the compounds and composition of the present disclosure can be released from the dosage form over an extended period of time. In certain embodiments, sustained release oral dosage forms can provide a therapeutically effective amount of a compound of the present disclosure over a period of at least several hours. In certain embodiments the extended release dosage form can provide a constant therapeutically effective concentration of a compound of the present disclosure in the plasma of a subject for a prolonged period of time, such as at least several hours. In other embodiments, the sustained release oral dosage form can provide a controlled and constant concentration of a therapeutically effective amount of a compound of the present disclosure in the plasma of a subject.

Dosage forms comprising compositions and chemical entities of the present disclosure can be administered at certain intervals such as, for example, twice per day or once per day.

Exemplary dosage ranges for oral administration are dependent on the potency of the compound of the present disclosure, but can range from 0.1 mg to 20 mg of the compound per kilogram of body weight. Dosage ranges may be readily determined by methods known to those skilled in the art.

Also provided are packaged pharmaceutical formulations. Such packaged formulations include a pharmaceutical composition comprising at least one chemical entity of the present disclosure, and instructions for using the composition to treat a mammal (typically a human patient). In some embodiments, the instructions are for using the pharmaceutical composition to treat a patient suffering from a disease responsive to inhibition at least one ATP-utilizing enzyme, such as a human protein kinase, for example AURORA-A, CK2, FLT3, c-KIT, PDGFR-α, PDGFR-β, GSK3-α, PDK1 and c-TAK1. Also provided is prescribing information; for example, to a patient or health care provider, or as a label in a packaged pharmaceutical formulation. Prescribing information may include for example efficacy, dosage and administration, contraindication and adverse reaction information pertaining to the pharmaceutical formulation.

Chemical entities of the present disclosure can be assayed in vitro and in vivo, to determine and optimize therapeutic or prophylactic activity prior to use in subjects. For example, in vitro assays can be used to determine whether administration of a specific compound of the present disclosure or a combination of such compounds exhibits therapeutic efficacy. Chemical entities of the present disclosure can also be demonstrated to be effective and safe using animal model systems.

It is desirable that a therapeutically effective dose of a compound of the present disclosure provide therapeutic benefit without causing substantial toxicity. Toxicity of chemical entities of the present disclosure can be determined using standard pharmaceutical procedures and can be readily ascertained by the skilled artisan. The dose ratio between toxic and therapeutic effect is the therapeutic index. In certain embodiments, chemical entities of the present disclosure can exhibit particularly high therapeutic indices in treating diseases and disorders. In certain embodiments, the dosage of a compound of the present disclosure can be within a range of circulating concentration that exhibits therapeutic efficacy with limited or no toxicity.

### EXAMPLES

Embodiments of the present disclosure can be further defined by reference to the following examples, which describe in detail preparation of chemical entities of the present disclosure and assays for using chemical entities of the present disclosure. It will be apparent to those skilled in the art that many modifications, both to materials and methods, may be practiced without departing from the scope of the present disclosure.

In the examples below, the following abbreviations have the following meanings. If an abbreviation is not defined, it has its generally accepted meaning.

AcOH=acetic acid

Atm=atmosphere

ATP=adenosine triphosphate

Boc=tert-butyloxycarbonyl

br=broad

BSA=bovine serum albumin

d=doublet

Da=Dalton

dd=doublet of doublets

DIEA=N,N-diisopropylethylamine

DMF=N,N-dimethylformamide

DMSO=dimethylsulfoxide

DTT=(R,R)-dithiothreitol

EDTA=ethylenediaminetetraacetic acid

ESI=electrospray ionization

EtOAc=ethyl acetate

EtOH=ethanol

FMOC=fluorenylmethoxycarbonyl

g=gram

HCl=hydrochloric acid

h=hour

HEPES=[4-(2-hydroxyethyl)-1-piperazineethanesulfonic acid

HPLC=high performance liquid chromatography

HTS=high throughput screen

Hz=hertz

i-PrOH=isopropanol

J=coupling constant

kDa=kilodalton

K₂CO₃=potassium carbonate

L=liter

LC/MS=liquid chromatography/mass spectroscopy

M=molar

MeOH=methanol

MgSO₄=magnesium sulfate

MHz=megahertz

mg=milligram

min=minute

mL=milliliter

mm=millimeter

mmol=millimoles

mM=millimolar

MS=mass spectroscopy

m/z=mass to charge ratio

nM=nanomolar

NMR=nuclear magnetic resonance

NaHCO₃=sodium bicarbonate

NaOH=sodium hydroxide

NMP=N-methylpyrrolidinone

psi=pounds per square inch

RT=room temperature

s=singlet

t=triplet

TCB=trough circulating buffer

THF=tetrahydrofuran

TFA=trifluoroacetic acid

TLC=thin layer chromatography

TMS=trimethylsilyl

UV=ultraviolet

v/v=volume to volume

W=watt

μL=microliter

μM=micromolar

### Example 1

### N-(4-(5-Phenyloxazol-2-yl)phenyl)acetamide

A mixture of 2-aminoacetophenone hydrochloride (12 mg, 0.072 mmol), 4-acetamidobenzoyl chloride (14 mg, 0.072 mmol), and N,N-diisopropylethylamine (63 μL, 0.36 mmol) in chloroform (1 mL) was stirred at room temperature for 2 h, then the solvents were evaporated in vacuo. The resulting residue was dissolved in dry dioxane (1 mL) followed by the addition of concentrated H₂SO₄ (30 μL). The reaction mixture was heated at 90° C. for 1 h, cooled to room temperature, then neutralized with 8M aqueous KOH (70 μL) and concentrated in vacuo. The resulting residue was suspended in DMSO (300 μl) and filtered. The filtrate was subjected to HPLC purification (Method 2) to provide N-(4-(5-phenyloxazol-2-yl)phenyl)acetamide (1 mg). LC/MS (ESI) m/z 279.1 [M+H]. HPLC retention time (Method A)=2.98 min.

### Example 2

### 2-Methyl-6-(5-(pyridin-3-yl)oxazol-2-yl)-1H-benzo[d]imidazole

A mixture of 2-bromo-1-(pyridin-3-yl)ethanone hydrobromide (1 g, 3.6 mmol) and hexamethylenetetramine (1.1 g, 7.9 mmol) in acetone (20 mL) was stirred overnight at room temperature. The formed precipitate was filtered, washed with ether (3×20 mL) and dried in vacuo. The crude complex was dissolved in 95% ethanol (20 mL), concentrated HCl (8 mL) was added, and the mixture was heated at reflux for 1 h, then cooled to room temperature. The formed precipitate was filtered and washed with ether (3×20 mL). The crude product was dissolved in water (5 mL) and subjected to HPLC purification (Method 1) to provide 2-amino-1-(pyridin-3-yl)ethanone trifluoroacetate (747 mg) as a brownish solid. LC/MS (ESI) m/z 137 [M+H]. HPLC retention time (Method A)=0.72 min.

A mixture of 2-amino-1-(pyridin-3-yl)ethanone trifluoroacetate (10 mg, 0.072 mmol), 2-methyl-3H-benzoimidazole-5-carboxylic acid (13 mg, 0.072 mmol), tris(dimethylamino)chlorophosphonium hexafluorophosphate (25 mg, 0.076 mmol) and N,N-diisopropylethylamine (63 μL, 0.36 mmol) in chloroform (1 mL) was stirred at room temperature for 2 h, then the solvents were evaporated in vacuo. The resulting residue was dissolved in dry dioxane (1 mL) followed by the addition of concentrated H₂SO₄ (30 μL). The reaction mixture was heated at 90° C. for 1 h, cooled to room temperature, then neutralized with 8M aqueous KOH (70 μL) and concentrated in vacuo. The resulting residue was suspended in DMSO (300 μL) and filtered. The filtrate was subjected to HPLC purification (Method 2) to provide 2-methyl-6-(5-(pyridin-3-yl)oxazol-2-yl)-1H-benzo[d]imidazole (1 mg). LC/MS (ESI) m/z 277.1 [M+H]. HPLC retention time (Method A)=1.55 min.

### Example 3

### 6-(5-(4-Methoxyphenyl)oxazol-2-yl)benzo[d]thiazole

A mixture of 2-bromo-1-(4-methoxyphenyl)ethanone (68 mg, 0.3 mmol) and hexamethylenetetramine (93 mg, 0.66 mmol) in acetone (3 mL) was stirred overnight at room temperature. The formed precipitate was filtered, washed with ether (3×3 mL) and dried in vacuo. The crude complex was dissolved in 95% ethanol (2 mL) and concentrated HCl (600 μL), and the mixture was heated at reflux for 1 h, then cooled to room temperature. The formed precipitate was filtered, washed with ether (3×3 mL), and dried in vacuo to provide 2-amino-1-(4-methoxyphenyl)ethanone hydrochloride (34 mg) as a brownish solid. LC/MS (ESI) m/z 166.2 [M+H]. HPLC retention time (Method A)=1.54 min.

A mixture of 2-amino-1-(4-methoxyphenyl)ethanone hydrochloride (8 mg, 0.04 mmol), benzothiazole-6-carboxylic acid (7 mg, 0.04 mmol), tris(dimethylamino)chloro phosphonium hexafluorophosphate (48 mg, 0.14 mmol), and N,N-diisopropylethylamine (24 μL, 0.14 mmol) in NMP (600 μL) was stirred at room temperature overnight, then the solvents were evaporated in vacuo. The resulting residue was dissolved in acetic anhydride (400 μL) followed by the addition of TFA (100 μL). The reaction mixture was heated at 90° C. for 1 h, cooled to the room temperature, and concentrated in vacuo. The resulting residue was dissolved in DMSO (200 μl) and subjected to HPLC purification (Method 2) to provide 6-(5-(4-methoxyphenyl)oxazol-2-yl)benzo[d]thiazole (2 mg). LC/MS (ESI) m/z 309.1 [M+H]. HPLC retention time (Method A)=3.53 min.

### Example 4

### 1-Methyl-5-(5-phenyloxazol-2-yl)-1H-benzo[d][1,2,3]triazole

A mixture of 2-aminoacetophenone hydrochloride (5.1 mg, 0.03 mmol), 1-methyl-1H-1,2,3-benzotriazole-5-carbonyl chloride (6 mg, 0.03 mmol), and N,N-diisopropylethylamine (13 μL, 0.076 mmol) in chloroform (500 μL) was stirred at room temperature for 2 h, then the solvents were evaporated in vacuo. The resulting residue was dissolved in acetic anhydride (400 μL) followed by the addition of TFA (100 μL). The reaction mixture was heated at 90° C. for 1.5 h, cooled to the room temperature, and concentrated in vacuo. The resulting residue was dissolved in DMSO (200 μL) and subjected to HPLC purification (Method 2) to provide 1-methyl-5-(5-phenyloxazol-2-yl)-1H-benzo[d][1,2,3]triazole (2 mg). LC/MS (ESI) m/z 277.1 [M+H]. HPLC retention time (Method A)=3.14 min.

### Example 5

### 6-(5-Phenyloxazol-2-yl)quinoline

A mixture of 2-aminoacetophenone hydrochloride (5.1 mg, 0.03 mmol), quinoline-6-carboxylic acid (6 mg, 0.03 mmol), tris(dimethylamino)chloro-phosphonium hexafluorophosphate (10 mg, 0.03 mmol), and N,N-diisopropylethylamine (16 μL, 0.09 mmol) in NMP (300 μL) was stirred at room temperature for 2 h, then the solvents were evaporated in vacuo. The resulting residue was dissolved in acetic anhydride (400 μL) followed by the addition of TFA (100 μL). The reaction mixture was heated at 90° C. for 1.5 h, cooled to the room temperature, and concentrated in vacuo. The resulting residue was dissolved in DMSO (200 μL) and subjected to HPLC purification (Method 2) to provide 6-(5-phenyloxazol-2-yl)quinoline (1 mg). LC/MS (ESI) m/z 273.1 [M+H]. HPLC retention time (Method A)=2.69 min.

### Example 6

### 2-Methyl-6-(5-phenyloxazol-2-yl)-1H-benzo[d]imidazole

A mixture of 2-aminoacetophenone hydrochloride (1.1 g, 6.4 mmol), 2-methyl-3H-benzoimidazole-5-carboxylic acid (1 g, 5.7 mmol), and N,N-diisopropylethyl amine (2.5 mL, 14.4 mmol) in chloroform (20 mL) was stirred at room temperature overnight. Solvents were evaporated in vacuo. The resulting residue was dissolved in EtOAc (80 mL) and washed with 5% aqueous NaHCO₃ and brine, dried over MgSO₄, and concentrated in vacuo to provide 2-methyl-N-(2-oxo-2-phenylethyl)-3H-benzoimidazole-5-carboxamide as a crude orange solid. LC/MS (ESI) m/z 294.2 [M+H]. HPLC retention time (Method A)=1.88 min.

A solution of 2-methyl-N-(2-oxo-2-phenylethyl)-3H-benzoimidazole-5-carboxamide (used crude from above) in POCl₃ (6 mL) was stirred at 100° C. for 1 h. The solvent was evaporated in vacuo. The resulting residue was dissolved in DMSO (2 mL) and subjected to HPLC purification (Method 1). The purified residue was dissolved in MeOH (1 mL), and 1M HCl in ether (50 mL) was added. The resulting precipitate was filtered and dried in vacuo to 2-methyl-6-(5-phenyloxazol-2-yl)-1H-benzo[d]imidazole (332 mg) as an off-white solid as the hydrochloride salt. LC/MS (ESI) m/z 276.3 [M+H]. HPLC retention time (Method A)=2.35 min.

### Example 7

### 5-(5-phenyloxazol-2-yl)-1H-benzo[d]imidazole hydrochloride

A mixture of 2-aminoacetophenone (1 g, 5.8 mmol), 5-benzimidazolecarboxylic acid (945 mg, 5.8 mmol), tris(dimethylamino)chlorophosphonium hexafluorophosphate (2.19 g, 6.38 mmol), and N,N-diisopropylethylamine (2.5 mL, 15 mmol) in chloroform (20 mL) was stirred at room temperature overnight, washed with 5% aqueous NaHCO₃, dried over Na₂SO₄, then concentrated to provide the crude amide, which was used immediately in the next step.

The crude amide prepared above was taken up in POCl₃ (20 mL) and heated at reflux for 1 h. After cooling to room temperature, the excess POCl₃ was evaporated, and the residue was partitioned between water (20 mL) and EtOAc (20 mL). The aqueous layer was basified to pH 9-10 with NaOH, and additional EtOAc (80 mL) was added. The organic layer was washed with 5% aqueous NaHCO₃ and brine, then dried over Na₂SO₄. Evaporation provided a crude residue, which was purified by preparative HPLC (Method 1). The resulting product was dissolved in CH₂Cl₂ and treated with an excess of HCl in Et₂O. Filtration provided the title compound (311 mg) as a white solid. LC/MS (ESI) m/z 261.9 [M+H]. HPLC retention time (Method A)=2.57 min.

### Example 8

### 5-(5-phenyloxazol-2-yl)benzo[d]thiazole

A mixture of 2-aminoacetophenone (860 mg, 5 mmol), 5-benzothiazolecarboxylic acid (880 mg, 4.91 mmol), tris(dimethylamino)chlorophosphonium hexafluorophosphate (1.72 g, 5 mmol), and N,N-diisopropylethylamine (2.5 mL, 15 mmol) in chloroform (20 mL) was stirred at room temperature overnight, washed with 5% aqueous NaHCO₃ and brine, dried over Na₂SO₄, then concentrated to provide the crude amide, which was used immediately in the next step.

The crude amide prepared above was taken up in POCl₃ (20 mL) and heated at reflux for 1 h. After cooling to room temperature, the excess POCl₃ was evaporated, and the residue was partitioned between water (20 mL) and EtOAc (20 mL). The aqueous layer was basified to pH 9-10 with NaOH, and additional EtOAc (80 mL) was added. The organic layer was washed with 5% aqueous NaHCO₃ and brine, then dried over Na₂SO₄. Evaporation provided a crude residue, which was purified by flash chromatography on silica gel, eluting with a gradient of 0% to 25% EtOAc in hexanes to give the title compound (1 g) as a white solid. LC/MS (ESI) m/z 279.1 [M+H]. HPLC retention time (Method A)=3.68 min.

### Example 9

### Characterization of Compounds

The following analytical HPLC condition was used for characterizing chemical entities of the present disclosure. MS ions were detected using a Sciex API-100 electrospray single quadrupole mass spectrometer interfaced to the HPLC system.

Method A: Phenomenex Chromolith SpeedRod RP-18e C18 analytical column (4.6 mm×50 mm); flow rate=1.5 mL/min; injection volume=15-20 μL; mobile phase A: 100% water, 0.1% trifluoroacetic acid (TFA); mobile phase B: 100% acetonitrile, 0.1% trifluoroacetic acid (TFA); gradient elution from 5% B to 100% B over 4.2 min, with a stay at 100% B for 1 min, then equilibration to 5% B over 0.8 min.

The following preparative HPLC methods were used for purifying chemical entities of the present disclosure:

Method 1: Nanosyn-Pack Microsorb 100-10 C-18 column (50 mm×300 mm); flow rate=100 mL/min; mobile phase A: 100% water, 0.1% trifluoroacetic acid (TFA); mobile phase B: 100% acetonitrile, 0.1% TFA; gradient elution from 0% B to 40% B over 90 min.

Method 2: Phenomenex Synergi 4 μm Max-RP column (10 mm×50 mm); flow rate=6 mL/min; mobile phase A: 100% water, 0.1% trifluoroacetic acid (TFA); mobile phase B: 100% acetonitrile, 0.1% trifluoroacetic acid (TFA); gradient elution from 10% B to 100% B over 8 min.

Method 3: Phenomenex Chromolith SpeedRod RP-18e C18 prep column (4.6 mm×50 mm); flow rate=4 mL/min; injection volume=60 μL; mobile phase A: 100% water, 0.1% trifluoroacetic acid (TFA); mobile phase B: 100% acetonitrile, 0.12% trifluoroacetic acid (TFA); gradient elution from 0% B to 100% B over 2.7 min, with a stay at 100% B for 0.6 min, then equilibration to 0% B over 0.7 min.

### Example 10

The following compounds are prepared by the general procedures as exemplified in the examples, utilizing the appropriate starting materials.

| | LC/MS | HPLC | | |
|---|---|---|---|---|
| | m/z | retention | HPLC | Synthetic |
| ChemDraw 8.0 Name | [M + H] | time (min) | Method | Procedure |
| 3-(2-phenyloxazol-5- | 223.1 | 2.11 | A | Example 1 |
| yl)pyridine | | | | |
| 2-(4-methoxybenzyl)-5- | 265.9 | 3.44 | A | Example 1 |
| phenyloxazole | | | | |
| 2-(4-methoxyphenyl)-5- | 252.3 | 3.67 | A | Example 1 |
| phenyloxazole | | | | |
| 3-(5-phenyloxazol-2- | 223.1 | 2.41 | A | Example 1 |
| yl)pyridine | | | | |
| 2-(3-methoxyphenyl)-5- | 252.3 | 3.72 | A | Example 1 |
| phenyloxazole | | | | |
| 3-(2-(3- | 253.1 | 2.23 | A | Example 1 |
| methoxyphenyl)oxazol-5- | | | | |
| yl)pyridine | | | | |
| N,N-dimethyl-4-(5- | 265.1 | 3.32 | A | Example 1 |
| phenyloxazol-2- | | | | |
| yl)benzenamine | | | | |
| methyl 4-(5-phenyloxazol- | 280.3 | 3.84 | A | Example 1 |
| 2-yl)benzoate | | | | |
| methyl 4-(5-(pyridin-3- | 281.1 | 2.29 | A | Example 1 |
| yl)oxazol-2-yl)benzoate | | | | |
| 2-(naphthalen-2-yl)-5- | 272.3 | 4.25 | A | Example 1 |
| phenyloxazole | | | | |
| 3-(2-(2- | 253.1 | 2.04 | A | Example 1 |
| methoxyphenyl)oxazol-5- | | | | |
| yl)pyridine | | | | |
| 3-((5-phenyloxazol-2- | 237.1 | 2.05 | A | Example 2 |
| yl)methyl)pyridine | | | | |
| 4-((5-phenyloxazol-2- | 237.1 | 2.05 | A | Example 2 |
| yl)methyl)pyridine | | | | |
| 2-(5-phenyloxazol-2- | 273.1 | 3.44 | A | Example 1 |
| yl)quinoline | | | | |
| 6-(5-phenyloxazol-2- | 279.1 | 3.65 | A | Example 2 |
| yl)benzo[d]thiazole | | | | |
| 2-((4- | 282.3 | 3.43 | A | Example 2 |
| methoxyphenoxy)methyl)- | | | | |
| 5-phenyloxazole | | | | |
| 4-(5-phenyloxazol-2- | 224.3 | 2.60 | A | Example 2 |
| yl)pyridazine | | | | |
| 3-((E)-2-(5-phenyloxazol-2- | 249.1 | 2.28 | A | Example 2 |
| yl)vinyl)pyridine | | | | |
| N,N-dimethyl-4-((E)-2-(5- | 291.1 | 2.83 | A | Example 2 |
| phenyloxazol-2- | | | | |
| yl)vinyl)benzenamine | | | | |
| 6-(5-(pyridin-3-yl)oxazol-2- | 275.1 | 1.94 | A | Example 1 |
| yl)quinoxaline | | | | |
| N-(4-(5-(pyridin-3- | 280.3 | 1.80 | A | Example 1 |
| yl)oxazol-2- | | | | |
| yl)phenyl)acetamide | | | | |
| 4-(5-phenyloxazol-2- | 280.3 | 3.49 | A | Example 1 |
| yl)phenyl acetate | | | | |
| 4-(5-(5-phenyloxazol-2- | 308.3 | 2.62 | A | Example 1 |
| yl)pyridin-2-yl)morpholine | | | | |
| 4-(5-(5-(pyridin-3- | 309.5 | 1.66 | A | Example 1 |
| yl)oxazol-2-yl)pyridin-2- | | | | |
| yl)morpholine | | | | |
| 2-(4-(N,N-di- | 385.1 | 4.17 | A | Example 1 |
| npropylsulfonyl)phenyl)-5- | | | | |
| phenyloxazole | | | | |
| 6-(5-(4- | 306.3 | 2.41 | A | Example 3 |
| methoxyphenyl)oxazol-2- | | | | |
| yl)-2-methyl-1H- | | | | |
| benzo[d]imidazole | | | | |
| 6-(5-(4- | 310.3 | 2.62 | A | Example 3 |
| chlorophenyl)oxazol-2-yl)- | | | | |
| 2-methyl-1H- | | | | |
| benzo[d]imidazole | | | | |
| 5-phenyl-2-p-tolyloxazole | 236.3 | 3.84 | A | Example 4 |
| 2-phenoxy-5-(5- | 315.1 | 3.77 | A | Example 4 |
| phenyloxazol-2-yl)pyridine | | | | |
| 2-chloro-4-(5-phenyloxazol- | 257.1 | 3.49 | A | Example 4 |
| 2-yl)pyridine | | | | |
| 2-(4-chlorophenyl)-5- | 256.3 | 3.96 | A | Example 4 |
| phenyloxazole | | | | |
| 2-(2-chlorophenyl)-5- | 256.3 | 3.72 | A | Example 4 |
| phenyloxazole | | | | |
| 3-(5-phenyloxazol-2- | 247.1 | 3.47 | A | Example 4 |
| yl)benzonitrile | | | | |
| N-(4-(5-phenyloxazol-2- | 280.3 | 2.62 | A | Example 5 |
| yl)pyridin-2-yl)acetamide | | | | |
| 2-chloro-5-(5-phenyloxazol- | 257.1 | 3.44 | A | Example 4 |
| 2-yl)pyridine | | | | |
| 2-(3-chlorophenyl)-5- | 256.3 | 3.97 | A | Example 4 |
| phenyloxazole | | | | |
| 2-(4-tert-butylphenyl)-5- | 278.3 | 4.28 | A | Example 4 |
| phenyloxazole | | | | |
| 5-(5-phenyloxazol-2- | 273.1 | 2.69 | A | Example 5 |
| yl)isoquinoline | | | | |
| 5-phenyl-2-o-tolyloxazole | 236.3 | 3.78 | A | Example 4 |
| 5-(5-phenyloxazol-2- | 239.1 | 2.25 | A | Example 5 |
| yl)pyrimidin-4-amine | | | | |
| 4-(3-(5-phenyloxazol-2- | 308.3 | 2.71 | A | Example 5 |
| yl)pyridin-2-yl)morpholine | | | | |
| 5-(5-phenyloxazol-2- | 273.1 | 2.81 | A | Example 5 |
| yl)quinoline | | | | |
| N-((5-phenyloxazol-2- | 280.3 | 2.16 | A | Example 5 |
| yl)methyl)nicotinamide | | | | |
| 3-(4-(5-phenyloxazol-2- | 306.3 | 2.69 | A | Example 5 |
| yl)thiazol-2-yl)pyridine | | | | |
| 5-phenyl-2-m-tolyloxazole | 236.3 | 3.78 | A | Example 4 |
| 1-(4-(5-phenyloxazol-2- | 287.9 | 3.57 | A | Example 4 |
| yl)phenyl)-1H-pyrazole | | | | |
| N-(6-(5-phenyloxazol-2- | 336.3 | 3.35 | A | Example 5 |
| yl)benzo[d]thiazol-2- | | | | |
| yl)acetamide | | | | |
| 5-(5-phenyloxazol-2-yl)- | 278.3 | 2.76 | A | Example 5 |
| 1H-benzo[d]imidazol- | | | | |
| 2(3H)-one | | | | |
| 5-(5-phenyloxazol-2-yl)- | 263.1 | 2.94 | A | Example 5 |
| 1H-benzo[d][1,2,3]triazole | | | | |
| 3-(5-phenyloxazol-2-yl)H- | 261.9 | 3.40 | A | Example 5 |
| pyrazolo[1,5-a]pyridine | | | | |
| (1H-indol-3-yl)(5- | 289.1 | 3.29 | A | Example 4 |
| phenyloxazol-2- | | | | |
| yl)methanone | | | | |
| 4-(5-phenyloxazol-2- | 247.1 | 3.51 | A | Example 4 |
| yl)benzonitrile | | | | |
| 6-chloro-2-(5-phenyloxazol- | 297.1 | 3.24 | A | Example 5 |
| 2-yl)imidazo[1,2- | | | | |
| b]pyridazine | | | | |

### Example 11

The following compounds are prepared by the general procedures as exemplified in the examples, utilizing the appropriate starting materials.

| | | HPLC | |
|---|---|---|---|
| | LC/MS m/z | retention | HPLC |
| ChemDraw 8.0 Name | [M + H] | time (min) | Method |
| 5-(4-bromophenyl)-2-(thiophen-2- | 305.9 | 2.38 | 3 |
| yl)oxazole | | | |
| 4-(5-(4-bromophenyl)oxazol-2- | 301.5 | 2.55 | 3 |
| yl)pyridine | | | |
| 3-(5-(4-methoxyphenyl)oxazol-2- | 253.1 | 1.66 | 3 |
| yl)pyridine | | | |
| 4-(5-(4-iodophenyl)oxazol-2- | 349.1 | 1.72 | 3 |
| yl)pyridine | | | |
| 4-(5-(4-methoxyphenyl)oxazol-2- | 303.1 | 1.83 | 3 |
| yl)quinoline | | | |
| 4-(5-(4-methoxyphenyl)oxazol-2- | 296.3 | 2.07 | 3 |
| yl)benzoic acid | | | |
| 5-phenyl-2-(thiophen-2-yl)oxazole | 228.3 | 2.25 | 3 |
| 4-(5-phenyloxazol-2-yl)quinoline | 273.1 | 1.90 | 3 |
| 3-(5-phenyloxazol-2-yl)-2H- | 289.9 | 2.13 | 3 |
| chromen-2-one | | | |
| 2,5-diphenyloxazole | 222.3 | 3.71 | A |
| 5-phenyl-2-styryloxazole | 248.3 | 2.48 | 3 |
| 2-(4-(morpholinylsulfonyl)phenyl)- | 371.1 | 2.16 | 3 |
| 5-phenyloxazole | | | |
| 2-(2-phenyloxazol-5-yl)quinoline | 273.1 | 1.82 | 3 |
| 3-(2-(4-methoxyphenyl)oxazol-5- | 253.1 | 1.63 | 3 |
| yl)pyridine | | | |

### Example 12

The following compounds are prepared by the general procedures as exemplified in the examples, utilizing the appropriate starting materials.

| | LC/MS | HPLC | |
|---|---|---|---|
| | m/z | retention time | |
| ChemDraw 8.0 Name | [M + H] | (min) | HPLC Method |
| 2-(benzo[d][1,3]dioxol-6-yl)-5-(2- | 285.1 | 1.93 | 3 |
| fluorophenyl)-1,3,4-oxadiazole | | | |
| 2-(4-(benzyloxy)-3-methoxyphenyl)-5-styryl- | 385.1 | 2.38 | 3 |
| 1,3,4-oxadiazole | | | |
| 2-(benzo[d][1,3]dioxol-6-yl)-5-(furan-2-yl)- | 257.1 | 1.74 | 3 |
| 1,3,4-oxadiazole | | | |
| 2-(4-ethoxyphenyl)-5-(4-fluorophenyl)-1,3,4- | 285.1 | 2.07 | 3 |
| oxadiazole | | | |
| 4-(4-(5-(4-fluorophenyl)-1,3,4-oxadiazol-2- | 326.3 | 1.99 | 3 |
| yl)phenyl)morpholine | | | |
| 4-(5-(4-chlorophenyl)-1,3,4-oxadiazol-2-yl)- | 300.3 | 2.27 | 3 |
| N,N-dimethylbenzenamine | | | |
| 4-(4-(5-(4-methoxyphenyl)-1,3,4-oxadiazol-2- | 338.3 | 1.96 | 3 |
| yl)phenyl)morpholine | | | |
| 3-(5-(3-aminophenyl)-1,3,4-oxadiazol-2- | 253.3 | 1.07 | 3 |
| yl)benzenamine | | | |
| 4-(5-(4-aminophenyl)-1,3,4-oxadiazol-2- | 253.1 | 1.25 | 3 |
| yl)benzenamine | | | |
| 4-(5-(3-methoxyphenyl)-1,3,4-oxadiazol-2- | 267.9 | 1.70 | 3 |
| yl)benzenamine | | | |
| 4-(5-(4-(difluoromethylsulfonyl)phenyl)-1,3,4- | 379.9 | 2.03 | 3 |
| oxadiazol-2-yl)-N,N-dimethylbenzenamine | | | |
| 4-(5-(4-ethoxyphenyl)-1,3,4-oxadiazol-2- | 268.3 | 1.45 | 3 |
| yl)pyridine | | | |
| N,N-dimethyl-4-(5-(pyridin-3-yl)-1,3,4- | 267.1 | 1.50 | 3 |
| oxadiazol-2-yl)benzenamine | | | |
| 4-(5-(4-butoxyphenyl)-1,3,4-oxadiazol-2- | 296.3 | 1.83 | 3 |
| yl)pyridine | | | |
| 4-(5-(4-isobutoxyphenyl)-1,3,4-oxadiazol-2- | 296.3 | 1.80 | 3 |
| yl)pyridine | | | |
| 4-(5-(3,4-dimethoxyphenyl)-1,3,4-oxadiazol-2- | 284.3 | 1.27 | 3 |
| yl)pyridine | | | |
| 4-(5-(2,4-dichlorophenyl)-1,3,4-oxadiazol-2- | 291.9 | 1.68 | 3 |
| yl)pyridine | | | |
| 4-(5-(pyridin-4-yl)-1,3,4-oxadiazol-2- | 239.1 | 1.03 | 3 |
| yl)benzenamine | | | |
| 4-(5-p-tolyl-1,3,4-oxadiazol-2-yl)pyridine | 237.9 | 1.52 | 3 |
| 4-(5-(2-chlorophenyl)-1,3,4-oxadiazol-2- | 258.3 | 1.52 | 3 |
| yl)pyridine | | | |
| 4-(5-(pyridin-4-yl)-1,3,4-oxadiazol-2- | 225.2 | 0.95 | 3 |
| yl)pyridine | | | |
| 4-(5-phenyl-1,3,4-oxadiazol-2-yl)benzoic acid | 267.1 | 1.79 | 3 |
| 2-(5-phenyl-1,3,4-oxadiazol-2-yl)benzenamine | 238.3 | 2.17 | 3 |
| 2,5-diphenyl-1,3,4-oxadiazole | 223.1 | 2.15 | 3 |
| 3-(5-(2-bromophenyl)-1,3,4-oxadiazol-2-yl)-7- | 440.3 | 2.16 | 3 |
| (diethylamino)-2H-chromen-2-one | | | |
| 3-(5-(2-bromophenyl)-1,3,4-oxadiazol-2-yl)-6- | 398.9 | 2.03 | 3 |
| methoxy-2H-chromen-2-one | | | |
| 3-(5-(furan-2-yl)-1,3,4-oxadiazol-2-yl)-1- | 266.3 | 2.05 | 3 |
| methyl-1H-indole | | | |
| 1-methyl-3-(5-(thiophen-2-yl)-1,3,4-oxadiazol- | 282.3 | 2.06 | 3 |
| 2-yl)-1H-indole | | | |
| 2-(3,4,5-trimethoxyphenyl)-5-(5-methylfuran- | 317.1 | 1.79 | 3 |
| 2-yl)-1,3,4-oxadiazole | | | |
| 2-(4-methoxyphenyl)-5-(5-methylfuran-2-yl)- | 257.1 | 1.93 | 3 |
| 1,3,4-oxadiazole | | | |
| ethyl 2-(4-(5-(thiophen-2-yl)-1,3,4-oxadiazol- | 331.5 | 1.95 | 3 |
| 2-yl)phenoxy)acetate | | | |
| 3-(4-(5-(furan-2-yl)-1,3,4-oxadiazol-2- | 328.3 | 1.43 | 3 |
| yl)phenylcarbamoyl)propanoic acid | | | |

Other embodiments of the present disclosure will be apparent to those skilled in the art from consideration of the specification and practice of the present disclosure disclosed herein. It is intended that the specification and examples be considered as exemplary only, with a true scope and spirit of the present disclosure being indicated by the following claims.

1. At least one chemical entity chosen from compounds of Formula I 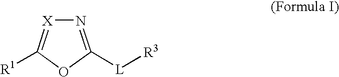
and pharmaceutically acceptable salts, solvates, chelates, non-covalent complexes, prodrugs, and mixtures thereof, wherein
R¹ is chosen from optionally substituted phenyl, optionally substituted furanyl, optionally substituted thienyl, optionally substituted pyridinyl, and optionally substituted quinolinyl;
X is CR²;
R² is hydrogen;
L is chosen from a covalent bond, —CH₂—, —CH═CH—, —CH₂O—, —CH₂NHC(O)—, and —C(O)—, and
R³ is chosen from optionally substituted aryl and optionally substituted heteroaryl, and
provided that
if R¹ is chosen from optionally substituted phenyl and pyridinyl, and L is chosen from a covalent bond and —CH₂—, then R³ is not chosen from
optionally substituted benzo[d][1,3]dioxolyl,
optionally substituted 1,3-dioxoisoindolin-2-yl,
optionally substituted 1-oxophthalazin-2(1H)-yl,
optionally substituted 7-oxo-4,5,6,7-tetrahydroindazol-1-yl,
optionally substituted 5-oxo-5,6,7,8-tetrahydroquinolin-2-yl,
optionally substituted 1,3-dioxo-1,3-dihydroisobenzofuran-5-yl,
2,3′-biquinolin-4-yl;
2,2′-biquinolin-4-yl;
(isoquinolin-3-yl)quinolin-4-yl;
quinolin-4-yl;
2-methyl-3-hydroxy-quinolin-4-yl;
2-phenyl-quinolin-4-yl;
quinolin-2-yl;
quinolin-5-yl;
optionally substituted thieno[3,2-b]pyridin-2-yl),
optionally substituted thieno[2,3-b]pyridin-2-yl,
optionally substituted benzo[d][1,3]dioxole-5-yl,
optionally substituted 2-oxo-2H-chromen-3-yl, and
optionally substituted 2-oxo-1,2-dihydroquinolin-4-yl;
if R¹ is optionally substituted phenyl and L is chosen from —CH₂—, —CH═CH—, and —C(O)—, then R³ is not chosen from benzofuran-3-yl and benzo[d]oxazol-2-yl;
if R¹ is optionally substituted phenyl and L is —CH₂O—, then R³ is not quinolin-2-yl;
if R¹ is chosen from optionally substituted phenyl, pyridinyl, thiophenyl, and L is a covalent bond, then R³ is not 6,7-dichloro-3-(4-(pyrrolidin-1-yl)butylamino)quinoxalin-2-yl;
if R¹ is optionally substituted phenyl, and L is a covalent bond, then R³ is not 1H-benzimidazol-5-yl optionally substituted at the 2-position of the benzimidazole ring with a group chosen from optionally substituted cycloalkyl, optionally substituted aryl, optionally substituted heteroaryl, optionally substituted aralkyl, optionally substituted heteroaralkyl, optionally substituted heterocycloalkyl, hydroxyl, alkylthio, and alkylsulfonyl;
and the compound of Formula I is not chosen from
7-phenyl-3-(5-phenyloxazol-2-yl)-3H-oxazolo[3,2-a][1,3,5]triazine-2,4-dione;
5-(5-phenyloxazol-2-yl)isobenzofuran-1,3-dione;
2-(chroman-6-yl)-5-(pyridin-4-yl)oxazole;
2-(7-(3,4-dichlorophenyl)-5-methyl-4,7-dihydropyrazolo[1,5-a]pyrimidin-6-yl)-5-phenyloxazole;
2-(2-ethylthieno[3,2-d]pyrimidin-4-yl)-5-(4-methoxyphenyl)oxazole;
2-(2-ethylthieno[3,2-d]pyrimidin-4-yl)-5-phenyloxazole;
2-(1,3-dimethyl-1,2,3,4-tetrahydroquinazolin-6-yl)-5-phenyloxazole; and
ethyl 2-morpholino-4-phenyl-6-(5-phenyloxazol-2-yl)-7-propylpyrrolo[1,2-b]pyridazine-5-carboxylate.

2. At least one chemical entity of claim 1 wherein R³ is chosen from fused 9 or 10 membered heterobicyclic ring systems containing one, two, three, or four heteroatoms chosen from nitrogen, oxygen, and sulfur wherein at least one of the rings in the ring system is aromatic and wherein the ring system is optionally substituted with one, two, or three groups chosen from halo, cyano, hydroxy, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl.

3. At least one chemical entity of claim 1 wherein R³ is chosen from
phenyl,
pyridinyl,
phenyl substituted with one, two, or three groups chosen from halo, cyano, hydroxy, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl; and
pyridinyl substituted with one, two, or three groups chosen from halo, cyano, hydroxy, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl.

4. At least one chemical entity of claim 1 wherein the compound of Formula I is chosen from compounds of Formula II wherein 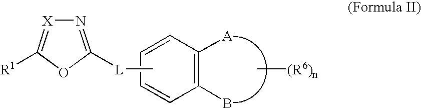
-A-B- is chosen from:
(a) —CH═CH—N═CH—,
(b) —CH═CH—CH═N—,
(c) —CH═CH—N═N—,
(d) —CH═N—N═CH—,
(e) —CH═N—CH═N—,
(f) —N═CH—CH═N—,
(g) —CH═CH—NH—,
(h) —CH═CH—O—,
(i) —CH═CH—S—,
(j) —N═CH—NH—,
(k) —CH═N—NH—
(l) —O—CH═N—,
(m) —CH═N—O—,
(n) —S—CH═N—,
(o) —CH═N—S—,
(p) —N═N—NH—,
(q) —CH₂—CH₂—CH═N—,
(r) —CH₂—CH₂—CH₂—NH—,
(s) —CH₂—CH₂—N═CH—,
(t) —CH₂—CH₂—NH—CH₂—,
(u) —CH₂—NH—C(O)—NH—,
(v) —CH₂—O—C(O)—NH—,
(w) —CH₂—NH—S(O)—NH—,
(x) —CH₂—NH—SO₂—NH—,
(y) —CH₂—CH₂—C(O)—NH—, and
(z) —CH═CH—C(O)—NH—;
n is chosen from 0, 1, 2, and 3; and
R⁶ (which may be on either or both of the rings of the heterobicyclic ring system) is chosen from halo, cyano, hydroxy, oxo, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl, and
wherein L is bound to the phenyl ring.

5. At least one chemical entity of claim 4 wherein -A-B- is chosen from
—N═CH—NH—,
—S—CH═N—,
—CH═CH—CH═N—,
—N═N—NH—,
—CH═CH—N═CH—, and
—N═CH—CH═N—.

6. At least one chemical entity of claim 1 wherein the compound of Formula I is chosen from compounds of Formula III wherein 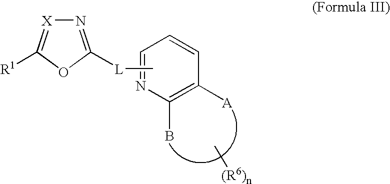
-A-B- is chosen from:
(a) —CH═CH—N═CH—,
(b) —CH═CH—CH═N—,
(c) —CH═CH—N═N—,
(d) —CH═N—N═CH—,
(e) —CH═N—CH═N—,
(f) —N═CH—CH═N—,
(g) —CH═CH—NH—,
(h) —CH═CH—O—,
(i) —CH═CH—S—,
(j) —N═CH—NH—,
(k) —CH═N—NH—
(l) —O—CH═N—,
(m) —CH═N—O—,
(n) —S—CH═N—,
(o) —CH═N—S—,
(p) —N═N—NH—,
(q) —CH₂—CH₂—CH═N—,
(r) —CH₂—CH₂—CH₂—NH—,
(s) —CH₂—CH₂—N═CH—,
(t) —CH₂—CH₂—NH—CH₂—,
(u) —CH₂—NH—C(O)—NH—,
(v) —CH₂—O—C(O)—NH—,
(w) —CH₂—NH—S(O)—NH—,
(x) —CH₂—NH—SO₂—NH—,
(y) —CH₂—CH₂—C(O)—NH—,
(z) —CH═CH—C(O)—NH—, and
(aa) —CH═CH—CH═CH—,
n is chosen from 0, 1, 2, and 3; and
R⁶ (which may be on either or both of the rings of the heterobicyclic ring system) is chosen from halo, cyano, hydroxy, oxo, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl and
wherein L is bound to the pyridinyl ring.

7. At least one chemical entity of claim 6 wherein -A-B- is —CH═CH—CH═CH—.

8. At least one chemical entity of claim 1 wherein the compound of Formula I is chosen from compounds of Formula IV wherein 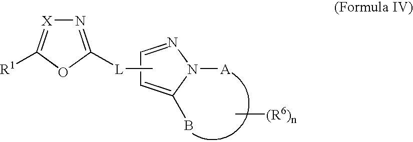
A-B- is chosen from:
(a) —CH═CH—N═CH—,
(b) —CH═CH—CH═N—,
(c) —CH═CH—N═N—,
(d) —CH═N—N═CH—,
(e) —CH═N—CH═N—,
(f) —N═CH—CH═N—,
(g) —CH═CH—NH—,
(h) —CH═CH—O—,
(i) —CH═CH—S—,
(j) —N═CH—NH—,
(k) —CH═N—NH—
(l) —O—CH═N—,
(m) —CH═N—O—,
(n) —S—CH═N—,
(o) —CH═N—S—,
(p) —N═N—NH—,
(q) —CH₂—CH₂—CH═N—,
(r) —CH₂—CH₂—CH₂—NH—,
(s) —CH₂—CH₂—N═CH—,
(t) —CH₂—CH₂—NH—CH₂—,
(u) —CH₂—NH—C(O)—NH—,
(v) —CH₂—O—C(O)—NH—,
(w) —CH₂—NH—S(O)—NH—,
(x) —CH₂—NH—SO₂—NH—,
(y) —CH₂—CH₂—C(O)—NH—,
(z) —CH═CH—C(O)—NH—;
(aa) —CH═CH—CH═CH—,
n is chosen from 0, 1, 2, and 3; and
R⁶ (which may be on either or both of the rings of the heterobicyclic ring system) is chosen from halo, cyano, hydroxy, oxo, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl, and
wherein L is bound to the pyrazole ring.

9. At least one chemical entity of claim 8 wherein -A-B- is —CH═CH—CH═CH—.

10. At least one chemical entity of claim 1 wherein the compound of Formula I is chosen from compounds of Formula V wherein 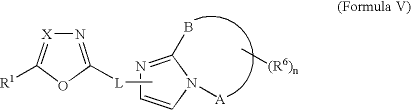
-A-B- is chosen from:
(a) —CH═CH—N═CH—,
(b) —CH═CH—CH═N—,
(c) —CH═CH—N═N—,
(d) —CH═N—N═CH—,
(e) —CH═N—CH═N—,
(f) —N═CH—CH═N—,
(g) —CH═CH—NH—,
(h) —CH═CH—O—,
(i) —CH═CH—S—,
(j) —N═CH—NH—,
(k) —CH═N—NH—
(l) —O—CH═N—,
(m) —CH═N—O—,
(n) —S—CH═N—,
(o) —CH═N—S—,
(p) —N═N—NH—,
(q) —CH₂—CH₂—CH═N—,
(r) —CH₂—CH₂—CH₂—NH—,
(s) —CH₂—CH₂—N═CH—,
(t) —CH₂—CH₂—NH—CH₂—,
(u) —CH₂—NH—C(O)—NH—,
(v) —CH₂—O—C(O)—NH—,
(w) —CH₂—NH—S(O)—NH—,
(x) —CH₂—NH—SO₂—NH—,
(y) —CH₂—CH₂—C(O)—NH—,
(z) —CH═CH—C(O)—NH—, and
(aa) —CH═CH—CH═CH—,
n is chosen from 0, 1, 2, and 3; and
R⁶ (which may be on either or both of the rings of the heterobicyclic ring system) is chosen from halo, cyano, hydroxy, oxo, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl and
wherein L is bound to the imidazole ring.

11. At least one chemical entity of claim 10 wherein -A-B- is —CH═CH—CH═N—.

12. At least one chemical entity of claim 1 wherein the compound of Formula I is chosen from compounds of Formula VI 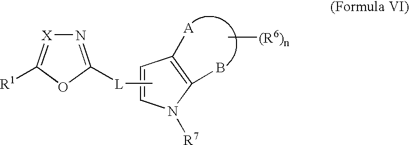
wherein -A-B- is chosen from:
(a) —CH═CH—N═CH—,
(b) —CH═CH—CH═N—,
(c) —CH═CH—N═N—,
(d) —CH═N—N═CH—,
(e) —CH═N—CH═N—,
(f) —N═CH—CH═N—,
(g) —CH═CH—NH—,
(h) —CH═CH—O—,
(i) —CH═CH—S—,
(j) —N═CH—NH—,
(k) —CH═N—NH—
(l) —O—CH═N—,
(m) —CH═N—O—,
(n) —S—CH═N—,
(o) —CH═N—S—,
(p) —N═N—NH—,
(q) —CH₂—CH₂—CH═N—,
(r) —CH₂—CH₂—CH₂—NH—,
(s) —CH₂—CH₂—N═CH—,
(t) —CH₂—CH₂—NH—CH₂—,
(u) —CH₂—NH—C(O)—NH—,
(v) —CH₂—O—C(O)—NH—,
(w) —CH₂—NH—S(O)—NH—,
(x) —CH₂—NH—SO₂—NH—,
(y) —CH₂—CH₂—C(O)—NH—,
(z) —CH═CH—C(O)—NH—; and
(aa) —CH═CH—CH═CH—,
n is chosen from 0, 1, 2, and 3; and
R⁶ (which may be on either or both of the rings of the heterobicyclic ring system) is chosen from halo, cyano, hydroxy, oxo, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl and,
R⁷ is chosen from hydrogen, optionally substituted alkyl, optionally substituted cycloalkyl, optionally substituted heterocycloalkyl, optionally substituted aryl, and optionally substituted heteroaryl,
wherein L is bound to the pyrrole ring.

13. At least one chemical entity of claim 1 wherein L is a covalent bond.

14. At least one chemical entity of claim 1 wherein the compound of Formula I is chosen from compounds of Formula VII 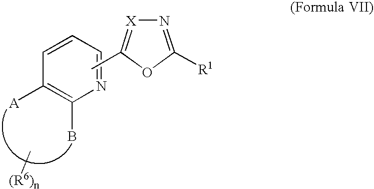
wherein -A-B- is chosen from:
(a) —CH═CH—N═CH—,
(b) —CH═CH—CH═N—,
(c) —CH═CH—N═N—,
(d) —CH═N—N═CH—,
(e) —CH═N—CH═N—,
(f) —N═CH—CH═N—,
(g) —CH═CH—NH—,
(h) —CH═CH—O—,
(i) —CH═CH—S—,
(j) —N═CH—NH—,
(k) —CH═N—NH—
(l) —O—CH═N—,
(m) —CH═N—O—,
(n) —S—CH═N—,
(o) —CH═N—S—,
(p) —N═N—NH—,
(q) —CH₂—CH₂—CH═N—,
(r) —CH₂—CH₂—CH₂—NH—,
(s) —CH₂—CH₂—N═CH—,
(t) —CH₂—CH₂—NH—CH₂—,
(u) —CH₂—NH—C(O)—NH—,
(v) —CH₂—O—C(O)—NH—,
(w) —CH₂—NH—S(O)—NH—,
(x) —CH₂—NH—SO₂—NH—,
(y) —CH₂—CH₂—C(O)—NH—,
(z) —CH═CH—C(O)—NH—, and
(aa) —CH═CH—CH═CH—,
n is chosen from 0, 1, 2, and 3; and
R⁶ (which may be on either or both of the rings of the heterobicyclic ring system) is chosen from halo, cyano, hydroxy, oxo, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl.

15. At least one chemical entity of claim 14 wherein -A-B- is —CH═CH—CH═CH—.

16. At least one chemical entity of claim 4 wherein n is 0.

17. At least one chemical entity of claim 4 wherein n is 1.

18. At least one chemical entity of claim 1 wherein R¹ is chosen from optionally substituted phenyl and optionally substituted pyridinyl.

19. At least one chemical entity of claim 18 wherein R¹ is chosen from phenyl and pyridinyl, each of which is optionally substituted with one, two or three groups chosen from are selected from halo, cyano, hydroxy, carboxy, nitro, alkoxy, substituted alkoxy, alkyl, substituted alkyl, aryl, substituted aryl, cycloalkyl, substituted cycloalkyl, heterocycloalkyl, substituted heterocycloalkyl, sulfanyl, substituted sulfanyl, sulfinyl, substituted sulfinyl, amino, substituted amino, aminocarbonyl, substituted aminocarbonyl, sulfonyl, substituted sulfonyl, acyl, and substituted acyl.

20. At least one chemical entity of claim 19 wherein R¹ is chosen from phenyl and pyridinyl, each of which is optionally substituted with one, two or three groups chosen from are selected from optionally substituted lower alkyl, optionally substituted lower alkoxy, halo, hydroxy, and cyano.

21. At least one chemical entity of claim 20 wherein R¹ is chosen from phenyl and pyridinyl, each of which is optionally substituted with one, two or three groups chosen from lower alkyl, lower alkoxy, halo, hydroxy, and cyano.

22. At least one chemical entity of claim 21 wherein R¹ is chosen from phenyl and pyridinyl.

23. At least one chemical entity of claim 1 wherein the compound is an inhibitor of at least one ATP-utilizing enzyme.

24. At least one chemical entity of claim 23 wherein the at least one ATP-utilizing enzyme is chosen from a human protein kinase.

25. At least one chemical entity of claim 24 wherein the human protein kinase is chosen from AURORA-A, CK2, FLT3, c-KIT, PDGFR-α, PDGFR-β, GSK3-α, PDK1 and c-TAK1.

26. At least one chemical entity of claim 25 wherein the human protein kinase is chosen from FLT3, c-KIT, PDGFR-α, and PDGFR-β.

27. A pharmaceutical composition comprising at least one pharmaceutically acceptable vehicle, and a therapeutically effective amount of at least one chemical entity of claim 1.

28. The pharmaceutical composition of claim 27, wherein the at least one chemical entity is present in an amount effective for the treatment in a patient of at least one disease chosen from transplant rejection, osteoarthritis, rheumatoid arthritis, multiple sclerosis, diabetes, diabetic retinopathy, asthma, inflammatory bowel disease, renal disease cachexia, septic shock, lupus, diabetes mellitus, myasthenia gravis, psoriasis, dermatitis, eczema, seborrhea, Alzheimer's disease, Parkinson's disease, stem cell protection during chemotherapy, ex vivo selection or ex vivo purging for autologous or allogeneic bone marrow transplantation, leukemia, cancer, ocular disease, corneal disease, glaucoma, bacterial infections, viral infections, fungal infections, heart disease, stroke, obesity, endometriosis, atherosclerosis, vein graft stenosis, peri-anastomatic prosthetic graft stenosis, prostate hyperplasia, chronic obstructive pulmonary disease, inhibition of neurological damage due to tissue repair, scar tissue formation, wound healing, pulmonary disease, neoplasm, and macular degeneration.

29. The pharmaceutical composition of claim 28, wherein cancer is chosen from at least one of glioblastoma, ovarian cancer, breast cancer, endometrial carcinoma, hepatocellular carcinoma, melanoma, colorectal cancer, colon cancer, digestive tract, lung cancer, renal-cell carcinoma, thyroid, lymphoid, prostate cancer and pancreatic cancer, etc., advanced tumors, hairy cell leukemia, melanoma, chronic myelogenous leukemia, advanced bead and neck, metastatic renal cell, non-Hodgkin's lymphoma, metastatic breast, breast adenocarcinoma, advanced melanoma, pancreatic, gastric, non-small cell lung, small cell lung, renal cell carcinoma, various solid tumors, multiple myeloma, metastatic prostate, malignant glioma, renal cancer, lymphoma, refractory metastatic disease, refractory multiple myeloma, cervical cancer, Kaposi's sarcoma, recurrent anaplastic glioma, and metastatic colon cancer.

30. The pharmaceutical composition of claim 28, wherein cancer is chosen from at least one of breast cancer, lung cancer, colorectal cancer, ovary cancer, prostate cancer, renal cancer, squamous cell cancer, glioblastoma, melanoma, pancreatic cancer, and Kaposi's sarcoma.

31. The pharmaceutical composition of claim 27 further comprising at least one additional therapeutic agent appropriate for effecting combination therapy.

32. A method of treating at least one disease in a patient in need of such treatment comprising administering to the patient a therapeutically effective amount of at least one chemical entity of claim 1 or at least one chemical entity chosen from
5-(5-phenyloxazol-2-yl)benzo[d]thiazole;
(1H-indol-3-yl)(5-phenyloxazol-2-yl)methanone;
1-(4-(5-phenyloxazol-2-yl)phenyl)-1H-pyrazole;
2-((4-methoxyphenoxy)methyl)-5-phenyloxazole;
2-(2-chlorophenyl)-5-phenyloxazole;
2-(2-phenyloxazol-5-yl)quinoline;
2-(3-chlorophenyl)-5-phenyloxazole;
2-(3-methoxyphenyl)-5-phenyloxazole;
2-(4-(morpholinylsulfonyl)phenyl)-5-phenyloxazole;
2-(4-(N,N-di-npropylsulfonyl)phenyl)-5-phenyloxazole;
2-(4-chlorophenyl)-5-phenyloxazole;
2-(4-methoxybenzyl)-5-phenyloxazole;
2-(4-methoxyphenyl)-5-phenyloxazole;
2-(4-tert-butylphenyl)-5-phenyloxazole;
2-(5-phenyloxazol-2-yl)quinoline;
2-(naphthalen-2-yl)-5-phenyloxazole;
2,5-diphenyloxazole;
2-chloro-4-(5-phenyloxazol-2-yl)pyridine;
2-chloro-5-(5-phenyloxazol-2-yl)pyridine;
2-phenoxy-5-(5-phenyloxazol-2-yl)pyridine;
3-((5-phenyloxazol-2-yl)methyl)pyridine;
3-((E)-2-(5-phenyloxazol-2-yl)vinyl)pyridine;
3-(2-(2-methoxyphenyl)oxazol-5-yl)pyridine;
3-(2-(3-methoxyphenyl)oxazol-5-yl)pyridine;
3-(2-(4-methoxyphenyl)oxazol-5-yl)pyridine;
3-(2-phenyloxazol-5-yl)pyridine;
3-(4-(5-phenyloxazol-2-yl)thiazol-2-yl)pyridine;
3-(5-(4-methoxyphenyl)oxazol-2-yl)pyridine;
3-(5-phenyloxazol-2-yl)-2H-chromen-2-one;
3-(5-phenyloxazol-2-yl)benzonitrile;
3-(5-phenyloxazol-2-yl)H-pyrazolo[1,5-a]pyridine;
3-(5-phenyloxazol-2-yl)pyridine;
4-((5-phenyloxazol-2-yl)methyl)pyridine;
4-(3-(5-phenyloxazol-2-yl)pyridin-2-yl)morpholine;
4-(5-(4-bromophenyl)oxazol-2-yl)pyridine;
4-(5-(4-iodophenyl)oxazol-2-yl)pyridine;
4-(5-(4-methoxyphenyl)oxazol-2-yl)benzoic acid;
4-(5-(4-methoxyphenyl)oxazol-2-yl)quinoline;
4-(5-(5-(pyridin-3-yl)oxazol-2-yl)pyridin-2-yl)morpholine;
4-(5-(5-phenyloxazol-2-yl)pyridin-2-yl)morpholine;
4-(5-phenyloxazol-2-yl)benzonitrile;
4-(5-phenyloxazol-2-yl)phenyl acetate;
4-(5-phenyloxazol-2-yl)pyridazine;
4-(5-phenyloxazol-2-yl)quinoline;
5-(4-bromophenyl)-2-(thiophen-2-yl)oxazole;
5-(5-phenyloxazol-2-yl)-1H-benzo[d][1,2,3]triazole;
5-(5-phenyloxazol-2-yl)-1H-benzo[d]imidazol-2(3H)-one;
5-(5-phenyloxazol-2-yl)-1H-benzo[d]imidazole;
5-(5-phenyloxazol-2-yl)isoquinoline;
5-(5-phenyloxazol-2-yl)pyrimidin-4-amine;
5-(5-phenyloxazol-2-yl)quinoline;
5-phenyl-2-(thiophen-2-yl)oxazole;
5-phenyl-2-m-tolyloxazole;
5-phenyl-2-o-tolyloxazole;
5-phenyl-2-p-tolyloxazole;
5-phenyl-2-styryloxazole;
6-(5-(4-chlorophenyl)oxazol-2-yl)-2-methyl-1H-benzo[d]imidazole;
6-(5-(4-methoxyphenyl)oxazol-2-yl)-2-methyl-1H-benzo[d]imidazole;
6-(5-(pyridin-3-yl)oxazol-2-yl)quinoxaline;
6-(5-phenyloxazol-2-yl)benzo[d]thiazole;
6-chloro-2-(5-phenyloxazol-2-yl)imidazo[1,2-b]pyridazine;
methyl 4-(5-(pyridin-3-yl)oxazol-2-yl)benzoate;
methyl 4-(5-phenyloxazol-2-yl)benzoate;
N-((5-phenyloxazol-2-yl)methyl)nicotinamide;
N-(4-(5-(pyridin-3-yl)oxazol-2-yl)phenyl)acetamide;
N-(4-(5-phenyloxazol-2-yl)pyridin-2-yl)acetamide;
N-(6-(5-phenyloxazol-2-yl)benzo[d]thiazol-2-yl)acetamide;
N,N-dimethyl-4-((E)-2-(5-phenyloxazol-2-yl)vinyl)benzenamine;
N,N-dimethyl-4-(5-phenyloxazol-2-yl)benzenamine;
2-(benzo[d][1,3]dioxol-6-yl)-5-(2-fluorophenyl)-1,3,4-oxadiazole;
2-(4-(benzyloxy)-3-methoxyphenyl)-5-styryl-1,3,4-oxadiazole;
2-(benzo[d][1,3]dioxol-6-yl)-5-(furan-2-yl)-1,3,4-oxadiazole;
2-(4-ethoxyphenyl)-5-(4-fluorophenyl)-1,3,4-oxadiazole;
4-(4-(5-(4-fluorophenyl)-1,3,4-oxadiazol-2-yl)phenyl)morpholine;
4-(5-(4-chlorophenyl)-1,3,4-oxadiazol-2-yl)-N,N-dimethylbenzenamine;
4-(4-(5-(4-methoxyphenyl)-1,3,4-oxadiazol-2-yl)phenyl)morpholine;
3-(5-(3-aminophenyl)-1,3,4-oxadiazol-2-yl)benzenamine;
4-(5-(4-aminophenyl)-1,3,4-oxadiazol-2-yl)benzenamine;
4-(5-(3-methoxyphenyl)-1,3,4-oxadiazol-2-yl)benzenamine;
4-(5-(4-(difluoromethylsulfonyl)phenyl)-1,3,4-oxadiazol-2-yl)-N,N-dimethylbenzenamine;
4-(5-(4-ethoxyphenyl)-1,3,4-oxadiazol-2-yl)pyridine;
N,N-dimethyl-4-(5-(pyridin-3-yl)-1,3,4-oxadiazol-2-yl)benzenamine;
4-(5-(4-butoxyphenyl)-1,3,4-oxadiazol-2-yl)pyridine;
4-(5-(4-isobutoxyphenyl)-1,3,4-oxadiazol-2-yl)pyridine;
4-(5-(3,4-dimethoxyphenyl)-1,3,4-oxadiazol-2-yl)pyridine;
4-(5-(2,4-dichlorophenyl)-1,3,4-oxadiazol-2-yl)pyridine;
4-(5-(pyridin-4-yl)-1,3,4-oxadiazol-2-yl)benzenamine;
4-(5-p-tolyl-1,3,4-oxadiazol-2-yl)pyridine;
4-(5-(2-chlorophenyl)-1,3,4-oxadiazol-2-yl)pyridine;
4-(5-(pyridin-4-yl)-1,3,4-oxadiazol-2-yl)pyridine;
4-(5-phenyl-1,3,4-oxadiazol-2-yl)benzoic acid;
2-(5-phenyl-1,3,4-oxadiazol-2-yl)benzenamine;
2,5-diphenyl-1,3,4-oxadiazole;
3-(5-(2-bromophenyl)-1,3,4-oxadiazol-2-yl)-7-(diethylamino)-2H-chromen-2-one;
3-(5-(2-bromophenyl)-1,3,4-oxadiazol-2-yl)-6-methoxy-2H-chromen-2-one;
3-(5-(furan-2-yl)-1,3,4-oxadiazol-2-yl)-1-methyl-1H-indole;
1-methyl-3-(5-(thiophen-2-yl)-1,3,4-oxadiazol-2-yl)-1H-indole;
2-(3,4,5-trimethoxyphenyl)-5-(5-methylfuran-2-yl)-1,3,4-oxadiazole;
2-(4-methoxyphenyl)-5-(5-methylfuran-2-yl)-1,3,4-oxadiazole;
ethyl 2-(4-(5-(thiophen-2-yl)-1,3,4-oxadiazol-2-yl)phenoxy)acetate; and
3-(4-(5-(furan-2-yl)-1,3,4-oxadiazol-2-yl)phenylcarbamoyl)propanoic acid;
and pharmaceutically acceptable salts, solvates, chelates, non-covalent complexes, prodrugs, and mixtures thereof.

33. The method of claim 32 wherein the at least one chemical entity is present in an amount effective for the treatment in a patient of at least one disease chosen from transplant rejection, osteoarthritis, rheumatoid arthritis, multiple sclerosis, diabetes, diabetic retinopathy, asthma, inflammatory bowel disease, renal disease cachexia, septic shock, lupus, diabetes mellitus, myasthenia gravis, psoriasis, dermatitis, eczema, seborrhea, Alzheimer's disease, Parkinson's disease, stem cell protection during chemotherapy, ex vivo selection or ex vivo purging for autologous or allogeneic bone marrow transplantation, leukemia, cancer, ocular disease, corneal disease, glaucoma, bacterial infections, viral infections, fungal infections, heart disease, stroke, obesity, endometriosis, atherosclerosis, vein graft stenosis, peri-anastomatic prosthetic graft stenosis, prostate hyperplasia, chronic obstructive pulmonary disease, inhibition of neurological damage due to tissue repair, scar tissue formation, wound healing, pulmonary disease, neoplasm, and macular degeneration.

34. The method of claim 33, wherein cancer is chosen from at least one of glioblastoma, ovarian cancer, breast cancer, endometrial carcinoma, hepatocellular carcinoma, melanoma, colorectal cancer, colon cancer, digestive tract, lung cancer, renal-cell carcinoma, thyroid, lymphoid, prostate cancer and pancreatic cancer, etc. advanced tumors, hairy cell leukemia, melanoma, chronic myelogenous leukemia, advanced bead and neck. metastatic renal cell, non-Hodgkin's lymphoma, metastatic breast, breast adenocarcinoma. advanced melanoma. pancreatic, gastric, non-small cell lung, small cell lung, renal cell carcinoma. various solid tumors, multiple myeloma, metastatic prostate, malignant glioma. renal cancer, lymphoma. refractory metastatic disease, refractory multiple myeloma, cervical cancer, Kaposi's sarcoma, recurrent anaplastic glioma, and metastatic colon cancer.

35. The method of claim 34, wherein cancer is chosen from at least one of breast cancer, lung cancer, colorectal cancer, ovary cancer, prostate cancer, renal cancer, squamous cell cancer, glioblastoma, melanoma, pancreatic cancer, and Kaposi's sarcoma.

36. The method of claim 31, further comprising administering at least one additional therapeutic agent appropriate for effecting combination therapy.

37. A method of inhibiting at least one ATP-utilizing enzyme in a subject comprising administering to the subject at least one chemical entity of claim 1 or at least one chemical entity chosen from
5-(5-phenyloxazol-2-yl)benzo[d]thiazole;
(1H-indol-3-yl)(5-phenyloxazol-2-yl)methanone;
1-(4-(5-phenyloxazol-2-yl)phenyl)-1H-pyrazole;
2-((4-methoxyphenoxy)methyl)-5-phenyloxazole;
2-(2-chlorophenyl)-5-phenyloxazole;
2-(2-phenyloxazol-5-yl)quinoline;
2-(3-chlorophenyl)-5-phenyloxazole;
2-(3-methoxyphenyl)-5-phenyloxazole;
2-(4-(morpholinylsulfonyl)phenyl)-5-phenyloxazole;
2-(4-(N,N-di-npropylsulfonyl)phenyl)-5-phenyloxazole;
2-(4-chlorophenyl)-5-phenyloxazole;
2-(4-methoxybenzyl)-5-phenyloxazole;
2-(4-methoxyphenyl)-5-phenyloxazole;
2-(4-tert-butylphenyl)-5-phenyloxazole;
2-(5-phenyloxazol-2-yl)quinoline;
2-(naphthalen-2-yl)-5-phenyloxazole;
2,5-diphenyloxazole;
2-chloro-4-(5-phenyloxazol-2-yl)pyridine;
2-chloro-5-(5-phenyloxazol-2-yl)pyridine;
2-phenoxy-5-(5-phenyloxazol-2-yl)pyridine;
3-((5-phenyloxazol-2-yl)methyl)pyridine;
3-((E)-2-(5-phenyloxazol-2-yl)vinyl)pyridine;
3-(2-(2-methoxyphenyl)oxazol-5-yl)pyridine;
3-(2-(3-methoxyphenyl)oxazol-5-yl)pyridine;
3-(2-(4-methoxyphenyl)oxazol-5-yl)pyridine;
3-(2-phenyloxazol-5-yl)pyridine;
3-(4-(5-phenyloxazol-2-yl)thiazol-2-yl)pyridine;
3-(5-(4-methoxyphenyl)oxazol-2-yl)pyridine;
3-(5-phenyloxazol-2-yl)-2H-chromen-2-one;
3-(5-phenyloxazol-2-yl)benzonitrile;
3-(5-phenyloxazol-2-yl)H-pyrazolo[1,5-a]pyridine;
3-(5-phenyloxazol-2-yl)pyridine;
4-((5-phenyloxazol-2-yl)methyl)pyridine;
4-(3-(5-phenyloxazol-2-yl)pyridin-2-yl)morpholine;
4-(5-(4-bromophenyl)oxazol-2-yl)pyridine;
4-(5-(4-iodophenyl)oxazol-2-yl)pyridine;
4-(5-(4-methoxyphenyl)oxazol-2-yl)benzoic acid;
4-(5-(4-methoxyphenyl)oxazol-2-yl)quinoline;
4-(5-(5-(pyridin-3-yl)oxazol-2-yl)pyridin-2-yl)morpholine;
4-(5-(5-phenyloxazol-2-yl)pyridin-2-yl)morpholine;
4-(5-phenyloxazol-2-yl)benzonitrile;
4-(5-phenyloxazol-2-yl)phenyl acetate;
4-(5-phenyloxazol-2-yl)pyridazine;
4-(5-phenyloxazol-2-yl)quinoline;
5-(4-bromophenyl)-2-(thiophen-2-yl)oxazole;
5-(5-phenyloxazol-2-yl)-1H-benzo[d][1,2,3]triazole;
5-(5-phenyloxazol-2-yl)-1H-benzo[d]imidazol-2(3H)-one;
5-(5-phenyloxazol-2-yl)-1H-benzo[d]imidazole;
5-(5-phenyloxazol-2-yl)isoquinoline;
5-(5-phenyloxazol-2-yl)pyrimidin-4-amine;
5-(5-phenyloxazol-2-yl)quinoline;
5-phenyl-2-(thiophen-2-yl)oxazole;
5-phenyl-2-m-tolyloxazole;
5-phenyl-2-o-tolyloxazole;
5-phenyl-2-p-tolyloxazole;
5-phenyl-2-styryloxazole;
6-(5-(4-chlorophenyl)oxazol-2-yl)-2-methyl-1H-benzo[d]imidazole;
6-(5-(4-methoxyphenyl)oxazol-2-yl)-2-methyl-1H-benzo[d]imidazole;
6-(5-(pyridin-3-yl)oxazol-2-yl)quinoxaline;
6-(5-phenyloxazol-2-yl)benzo[d]thiazole;
6-chloro-2-(5-phenyloxazol-2-yl)imidazo[1,2-b]pyridazine;
methyl 4-(5-(pyridin-3-yl)oxazol-2-yl)benzoate;
methyl 4-(5-phenyloxazol-2-yl)benzoate;
N-((5-phenyloxazol-2-yl)methyl)nicotinamide;
N-(4-(5-(pyridin-3-yl)oxazol-2-yl)phenyl)acetamide;
N-(4-(5-phenyloxazol-2-yl)pyridin-2-yl)acetamide;
N-(6-(5-phenyloxazol-2-yl)benzo[d]thiazol-2-yl)acetamide;
N,N-dimethyl-4-((E)-2-(5-phenyloxazol-2-yl)vinyl)benzenamine;
N,N-dimethyl-4-(5-phenyloxazol-2-yl)benzenamine;
2-(benzo[d][1,3]dioxol-6-yl)-5-(2-fluorophenyl)-1,3,4-oxadiazole;
2-(4-(benzyloxy)-3-methoxyphenyl)-5-styryl-1,3,4-oxadiazole;
2-(benzo[d][1,3]dioxol-6-yl)-5-(furan-2-yl)-1,3,4-oxadiazole;
2-(4-ethoxyphenyl)-5-(4-fluorophenyl)-1,3,4-oxadiazole;
4-(4-(5-(4-fluorophenyl)-1,3,4-oxadiazol-2-yl)phenyl)morpholine;
4-(5-(4-chlorophenyl)-1,3,4-oxadiazol-2-yl)-N,N-dimethylbenzenamine;
4-(4-(5-(4-methoxyphenyl)-1,3,4-oxadiazol-2-yl)phenyl)morpholine;
3-(5-(3-aminophenyl)-1,3,4-oxadiazol-2-yl)benzenamine;
4-(5-(4-aminophenyl)-1,3,4-oxadiazol-2-yl)benzenamine;
4-(5-(3-methoxyphenyl)-1,3,4-oxadiazol-2-yl)benzenamine;
4-(5-(4-(difluoromethylsulfonyl)phenyl)-1,3,4-oxadiazol-2-yl)-N,N-dimethylbenzenamine;
4-(5-(4-ethoxyphenyl)-1,3,4-oxadiazol-2-yl)pyridine;
N,N-dimethyl-4-(5-(pyridin-3-yl)-1,3,4-oxadiazol-2-yl)benzenamine;
4-(5-(4-butoxyphenyl)-1,3,4-oxadiazol-2-yl)pyridine;
4-(5-(4-isobutoxyphenyl)-1,3,4-oxadiazol-2-yl)pyridine;
4-(5-(3,4-dimethoxyphenyl)-1,3,4-oxadiazol-2-yl)pyridine;
4-(5-(2,4-dichlorophenyl)-1,3,4-oxadiazol-2-yl)pyridine;
4-(5-(pyridin-4-yl)-1,3,4-oxadiazol-2-yl)benzenamine;
4-(5-p-tolyl-1,3,4-oxadiazol-2-yl)pyridine;
4-(5-(2-chlorophenyl)-1,3,4-oxadiazol-2-yl)pyridine;
4-(5-(pyridin-4-yl)-1,3,4-oxadiazol-2-yl)pyridine;
4-(5-phenyl-1,3,4-oxadiazol-2-yl)benzoic acid;
2-(5-phenyl-1,3,4-oxadiazol-2-yl)benzenamine;
2,5-diphenyl-1,3,4-oxadiazole;
3-(5-(2-bromophenyl)-1,3,4-oxadiazol-2-yl)-7-(diethylamino)-2H-chromen-2-one;
3-(5-(2-bromophenyl)-1,3,4-oxadiazol-2-yl)-6-methoxy-2H-chromen-2-one;
3-(5-(furan-2-yl)-1,3,4-oxadiazol-2-yl)-1-methyl-1H-indole;
1-methyl-3-(5-(thiophen-2-yl)-1,3,4-oxadiazol-2-yl)-1H-indole;
2-(3,4,5-trimethoxyphenyl)-5-(5-methylfuran-2-yl)-1,3,4-oxadiazole;
2-(4-methoxyphenyl)-5-(5-methylfuran-2-yl)-1,3,4-oxadiazole;
ethyl 2-(4-(5-(thiophen-2-yl)-1,3,4-oxadiazol-2-yl)phenoxy)acetate; and
3-(4-(5-(furan-2-yl)-1,3,4-oxadiazol-2-yl)phenylcarbamoyl)propanoic acid;
and pharmaceutically acceptable salts, solvates, chelates, non-covalent complexes, prodrugs, and mixtures thereof.

38. The method of claim 37 wherein the at least one ATP-utilizing enzyme is chosen from a human protein kinase.

39. The method of claim 38 wherein the human protein kinase is a tyrosine receptor kinase.

40. The method of claim 39 wherein the human protein kinase is chosen from wild-type and mutant PDGFR-α, PDGFR-β, FLT-3, and c-KIT receptors.

41. A packaged pharmaceutical formulation comprising a pharmaceutical composition of claim 27 and instructions for using the composition to treat a mammal.

42. The packaged pharmaceutical formulation of claim 41 wherein the instructions are for using the pharmaceutical composition to treat a patient suffering from a disease responsive to inhibition at least one ATP-utilizing enzyme.

43. The packaged pharmaceutical formulation of claim 42 wherein the human protein kinase is a tyrosine receptor kinase.

44. The packaged pharmaceutical formulation of claim 42 wherein the human protein kinase is chosen from wild-type and mutant PDGFR-α, PDGFR-β, FLT-3, and c-KIT receptors.
